(19) United States
(12) Patent Application Publication
(10) Pub. No.: US 2026/0062749 A1
(43) Pub. Date: Mar. 5, 2026

(54) A METHOD FOR DETERMINING THE TISSUE OR CELL OF ORIGIN OF DNA

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Yuval DOR, Jerusalem (IL); Ruth SHEMER, Mevasseret Zion (IL); Benjamin GLASER, Jerusalem (IL); Tomer KAPLAN, Jerusalem (IL)

(73) Assignees: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(21) Appl. No.: 19/104,414

(22) PCT Filed: Aug. 17, 2023
(86) PCT No.: PCT/IL2023/050870

§ 371 Date: Feb. 18, 2025

Related U.S. Application Data
(60) Provisional application No. 63/371,833, filed on Aug. 18, 2022.

(51) Int. Cl. C12Q 1/6881; C12Q 1/6806; C12Q 1/6827; C12Q 1/6844; C12Q 1/6869

(52) U.S. Cl. CPC C12Q 1/6881; C12Q 1/6806; C12Q 1/6827; C12Q 1/6844; C12Q 1/6869; C12Q 2600/154

(57) ABSTRACT

Methods of detecting death of a cell type or tissue in a subject comprising determining the origin of cfDNA by ascertaining the methylation status of two or three methylation sites on a cfDNA molecule of no more than 167 nucleotides is provided. Methods of identifying regions of genomic DNA comprising no more than 167 nucleotides whose methylation signature distinguishes the origin of the region are also provided. Kits for use in performing the methods of the invention are also provided.

### CROSS REFERENCE TO RELATED APPLICATIONS

This application claims the benefit of priority of U.S. Provisional Patent Application No. 63/371,833, filed on Aug. 18, 2022, the contents of which are all incorporated herein by reference in their entirety.

### REFERENCE TO AN ELECTRONIC SEQUENCE LISTING

The contents of the electronic sequence listing (HUJI-HDST-P-092-PCT.xml; Size: 69,240 bytes; and Date of Creation: Aug. 14, 2023) is herein incorporated by reference in its entirety.

### FIELD OF INVENTION

The present invention is in the field of DNA methylation analysis.

### BACKGROUND OF THE INVENTION

The methylation status of some cytosines adjacent to guanosines in the genome (CpG sites) is typical of specific cell types. Some sites are unmethylated in a specific cell type and methylated elsewhere, while others (much fewer) are methylated in a specific cell type and unmethylated elsewhere. Information on this status can inform on the tissue origins of DNA molecules, including within mixtures, such as cell-free DNA (cfDNA) circulating in plasma. Altered contribution of cfDNA from a specific tissue source is often indicative of pathology is that tissue and is an important emerging biomarker with clinical utility.

When searching for useful tissue-specific methylation markers, there are two key considerations. 1) Sensitivity: the marker should allow to capture as many molecules as possible from the tissue of interest. 2) Specificity: the marker should cause minimal “noise”, that is a false signal from DNA of other cell types.

In previous studies, it has been shown that by using the methylation status of multiple adjacent cytosines in the same molecule, the specificity for detection of DNA from a given tissue can be greatly enhanced compared to the use of individual CpG sites, without a major loss of sensitivity. International Patent Application WO2015159292 discloses the use of clusters of adjacent CpG sites, containing 4 or more CpGs, within molecules that are 50-300 nucleotides long, as tissue-specific biomarkers. This application demonstrated that information could only be gleaned when four or more CpGs were present, which limits the number of informative loci that exist. Superior methods of determining the tissue or cell type of origin of DNA are greatly needed.

### SUMMARY OF THE INVENTION

The present invention provides methods of detecting the tissue origin of cfDNA by ascertaining the methylation status of two or three methylation sites on the same cfDNA molecule of no more than 167 nucleotides (i.e., the peak size of cfDNA molecules, reflecting nucleosome size) is provided.

The present invention further provides methods of detecting death of a cell type or tissue in a subject comprising determining the origin of cfDNA by ascertaining the methylation status of two or three methylation sites on the same cfDNA molecule of no more than 167 nucleotides is provided.

Methods of identifying regions of genomic DNA comprising no more than 167 nucleotides whose methylation signature distinguishes the origin of the region are also provided. Kits for use in performing the methods of the invention are also provided.

According to a first aspect, there is provided a method of detecting the tissue origin of DNA in a subject comprising determining whether cell-free DNA (cfDNA) comprised in a fluid sample of the subject is derived from the cell type or tissue, wherein the determining is effected by ascertaining the methylation status of two or three methylation sites on a continuous sequence of the cell-free DNA, the sequence comprising no more than 167 nucleotides, wherein a methylation status of each of the two or three methylation sites on the continuous sequence of the DNA characteristic of the cell type or tissue is indicative of death of the cell type or tissue.

According to some embodiments, cfDNA from the cell type or tissue comprises more than 0.1% of the total cfDNA in the fluid sample.

According to some embodiments, the cell type or tissue is selected from a blood cell type, vascular endothelial cells and hepatocytes.

According to some embodiments, the cell type or tissue is selected from liver, lung, vascular endothelium, gastrointestinal tract, B cells, T cells, monocytes, neutrophils, natural killer (NK) cells and eosinophils.

According to another aspect, there is provided a method of identifying a subregion of genomic DNA comprising no more than 167 nucleotides whose methylation signature in a cell type or tissue of interest distinguishes it from a second non-identical cell type or tissue, the method comprising:
a. identifying in genomic DNA of the cell type or tissue of interest a plurality of methylation sites that are each differentially methylated with respect to the second non-identical cell type or tissue of interest;
b. selecting from the plurality of sites at least one site, wherein the at least one site is located in a region comprising at least 6 CpGs within 167 nucleotides upstream and 167 nucleotides downstream of the at least one site; and
c. identifying with the region of genomic DNA a subregion comprising no more than 167 nucleotides and 2 or 3 methylation sites wherein each of the 2 or 3 sites comprise the same methylation status and are differentially methylated with respect to the second non-identical cell type or tissue of interest;
thereby identifying a subregion of genomic DNA comprising no more than 167 nucleotides.

According to some embodiments:
a. the identifying a plurality of methylation sites comprises comparing methylation statuses of a plurality of methylation sites with a panel or atlas of methylation statuses for the plurality of methylation sites from genomic DNA extracted from a plurality of tissues and/or cell types;
b. the identifying a subregion comprises comparing the methylation status of the 2 or 3 methylation sites with a panel or atlas of methylation statuses for the 2 or 3 methylation sites from genomic DNA extracted from a plurality of tissues and/or cell types; or
c. both.

According to some embodiments, the second non-identical cell type or tissue of interest is selected from a blood cell type, vascular endothelial cells and hepatocytes.

According to some embodiments, the continuous sequence of the cell-free DNA comprises or consists of a subregion identified by a method of the invention.

According to some embodiments, the continuous sequence of cell-free DNA comprises or consists of a sequence selected from SEQ ID NO: 1-70.

According to some embodiments, the fluid is selected from the group consisting of blood, plasma, sperm, milk, urine, saliva and cerebral spinal fluid.

According to some embodiments, the sample is a blood sample.

According to some embodiments, the ascertaining is effected using at least one methylation-dependent oligonucleotide.

According to some embodiments, the ascertaining is effected by:
(a) contacting the DNA in the sample (e.g., with bisulfite) to convert unmethylated cytosines of the DNA to uracils;
(b) amplifying the continuous sequence of DNA using oligonucleotides that hybridize to a nucleic acid sequence adjacent to the first and last of the 2 or 3 methylation sites on the continuous sequence of the DNA; and
(c) sequencing the continuous sequence of DNA.

According to some embodiments, the sequencing is deep sequence, next generation sequencing or both.

According to some embodiments, the method further comprises quantitating the amount of cell-free DNA which is derived from the cell type or tissue.

According to some embodiments, the method is a computerized method.

According to another aspect, there is provided a system for identifying a subregion of genomic DNA comprising no more than 167 nucleotides whose methylation signature in a cell type or tissue of interest distinguishes it from a second non-identical cell type or tissue, the system comprising a non-transitory memory device, wherein modules of instruction code are stored, and at least one processor associated with the memory device, and configured to execute the modules of instruction code, whereupon execution of the modules of instruction code, the at least one processor is configured to:
identify in genomic DNA of the cell type or tissue of interest a plurality of methylation sites that are each differentially methylated with respect to the second non-identical cell type or tissue of interest;
select from the plurality of sites at least one site, wherein the at least one site is located in a region comprising at least 6 CpGs within 167 nucleotides upstream and 167 nucleotides downstream of the at least one site; and
identify with the region of genomic DNA a subregion comprising no more than 167 nucleotides and 2 or 3 methylation sites wherein each of the 2 or 3 sites comprise the same methylation status and are differentially methylated with respect to the second non-identical cell type or tissue of interest.

According to another aspect, there is provided a kit for identifying the source of DNA in a sample comprising oligonucleotides which are capable of detecting the methylation status of two or three methylation sites in a nucleic acid sequence, the nucleic acid sequence being no longer than 167 base pairs and comprising two or three methylation sites which are differentially methylated in a first cell of interest with respect to a second cell which is non-identical to the first cell of interest.

According to some embodiments, the nucleic acid sequence is comprised in a sequence as set forth in any one of SEQ ID NO: 1-70.

According to some embodiments, the kit further comprises at least one agent for sequencing the nucleic acid sequence, bisulfite or both.

According to some embodiments, the kit is for use in a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B: (1A) Bar graphs of vascular endothelial cell methylation markers demonstrating that 3 CpGs are as informative (i.e., equally or more sensitive, and as specific) as 4 or 5 CpGs. (1B) Table of the sequences of the regions probed in 1A and the locations of the CpGs therein. Chromosome positions are given within human genome build HG19. Cytosines of CpGs are underlined. The three informative cytosines are also bolded and marked in red.

FIGS. 2A-2H: (2A) Bar graphs of hepatocyte methylation markers demonstrating that 3 CpGs are as informative as 5 or 6 CpGs. (2B) Table of the sequences of the regions probed in 2A and the locations of the CpGs therein. Chromosome positions are given within human genome build HG19. Cytosines of CpGs are underlined. The three informative cytosines are also bolded and marked in red. (2C-2H) Bar graphs of (2C) colon, (2D) endothelial cell, (2E) heart, (2F) brain, (2G) pancreas and (2H) megakaryocyte methylation markers demonstrating that 3 CpGs are as informative as greater numbers of CpGs.

FIG. 3: A bar graph of % unmethylation by tissue using 7, 3, 2 or 1 CpG within a lung alveolar epithelium methylation marker (the RAB4 gene).

FIG. 4A-4C: (4A) A bar graph of % unmethylation within the RAB4 gene using 7, 3, 2 or 1 CpGs in healthy control individuals and individuals suffering from lung cancer or COPD. (4B) A bar graph of % unmethylation within liver markers using 1, 2, 3 or more CpGs in samples from liver transplant recipients experiencing rejection and healthy controls. (4C) A bar graph of % unmethylation within a colon marker using 1, 2, 3 or 6 CpGs in samples from bone marrow transplant recipients experiencing GVHD and healthy controls.

FIG. 5: Diagram of an embodiment of a method of the invention.

FIG. 6: Diagram of an embodiments of a computing device to be used in embodiments of the invention.

### DETAILED DESCRIPTION OF THE INVENTION

The present invention, in some embodiments thereof, relates to a method of determining the source of cell-free DNA and use thereof such as for diagnosing pathological processes associated with cell death, monitoring therapeutic regimes such as drugs intended to change cell death and in studying for clinical and research purposes processes affecting cell death levels.

The invention is based, at least in part, on the surprising finding that methylation patterns are determined in a regional manner during cell differentiation, such that multiple adjacent cytosines are affected when a methylase (e.g., DNMT) or demethylase (e.g., TET) acts on a particular sequence. In contrast, “accidents” of aberrant methylation or demethylation in irrelevant tissues, may take place in one cytosine but not in a cluster of multiple adjacent cytosines present on the same continuous stretch of nucleotides. It was surprisingly found that there are regions where as few as 2 or 3 CpGs can uniquely identify a tissue or cell type of origin with sufficiently low noise from other tissues/cells.

By a first aspect, there is provided a method of identifying a methylation signature for a cell type or tissue of interest comprising identifying in the DNA of the cell type or tissue of interest a continuous sequence of no more than 170, 167 or 150 nucleotides which comprise 2 or 3 methylation sites, wherein each of the sites are differentially methylated with respect to a second non-identical cell, thereby identifying the methylation signature for the cell type or tissue of interest.

In some embodiments, the method is an in vitro method. In some embodiments, the method is an ex vivo method. In some embodiments, the method is a diagnostic method. In some embodiments, the method is a method of diagnosing a pathology. In some embodiments, detection of cell death of a tissue or cell type is indicative of a pathology. In some embodiments, death of liver cells is indicative of liver transplant rejection. In some embodiments, death of cells is indicative of graft vs host disease (GVHD) in a subject after a bone marrow transplant. In some embodiments, the transplant is an allogenic transplant. In some embodiments, death of colon cells is indicative of GVHD.

The present invention contemplates identifying methylation signatures in any cell of interest, including but not limited to pancreatic cells (such as pancreatic beta cells, duct or acinar cells), brain cells, oligodendrocytes, cardiac cells (e.g., cardiomyocytes), liver cells (e.g., hepatocytes), kidney cells, vascular endothelial cells, lymphocytes, lung cells (e.g., alveolar epithelium cells), uterus cells, breast cells, adipocytes, colon cells, rectum cells, prostate cells, thyroid cells and skeletal muscle cells.

As used herein, the term “methylation site” refers to a cytosine residue adjacent to guanine residue (CpG site) that has a potential of being methylated.

The continuous sequence is preferably no longer than 170 nucleotides, 165 nucleotides, 160 nucleotides, 155 nucleotides, 150 nucleotides, 145 nucleotides, 140 nucleotides, 135 nucleotides, 130 nucleotides, 125 nucleotides, 120 nucleotides, 115 nucleotides, 110 nucleotides, 105 nucleotides, 100 nucleotides, 95 nucleotides, 90 nucleotides, 85 nucleotides, 80 nucleotides, 75 nucleotides, 70 nucleotides, 65 nucleotides, 60 nucleotides, 55 nucleotides, or 50 nucleotides. In some embodiments, the continuous sequence is no longer than 167 nucleotides.

In some embodiments, a continuous sequence is a sequence on a single DNA molecule. In some embodiments, a single DNA molecule is a single physical strand of DNA. In some embodiments, a single DNA molecule is a single physical DNA molecule. It will be understood that a sequence of a continuous sequence (i.e., single DNA molecule) is not a sequencing of a mix of DNA molecules that provides a consensus sequence (e.g., such as is produced by pyrosequencing) but rather is the sequence of a single continuous strand (e.g., such as is produced by next generation sequencing).

According to a particular embodiment, the sequence is between 50-170 nucleotides, e.g., between 50-150 nucleotides, between 50-100 nucleotides, between 70-170 nucleotides, between 90-170 nucleotides, between 100-170 nucleotides, or between 150-170 nucleotides.

The sequence may be of a coding or non-coding region. According to a particular embodiment, the sequence is not derived from a gene which is differentially expressed in the cell of interest. Thus, for example in the case of identifying a methylation pattern for a pancreatic beta cell, the DNA sequence may not be part of a gene encoding insulin or another pancreatic beta cell protein. In some embodiments, the sequence is derived from a gene which is differentially expressed in the cell of interest. In some embodiments, the sequence is derived from a regulatory region of a gene which is differentially expressed in the cell of interest.

In accordance with another particular embodiment, the methylation pattern characterizes the normal cell of interest and is not a methylation pattern characterizing a diseased cell (is not for example a methylation pattern characterizing cancer cells of a specific type).

In some embodiments, the continuous nucleic acid sequences comprise 2 methylation sites. In some embodiments, the continuous nucleic acid sequence comprises 3 methylation sites. In some embodiments, the continuous nucleic acid sequences comprise 4 or more methylation sites, and methylation status of 2 or 3 of those methylation sites are ascertained.

In order to be considered a methylation signature for a particular cell of interest each of the at two or three methylation sites have to be differentially methylated in that cell of interest with respect to a second non-identical cell. In some embodiments, the continuous sequence comprises or consists of a region identified by a method of the invention. In some embodiments, the continuous sequence comprises or consists of a subregion identified by a method of the invention.

According to a particular embodiment, each of the 2 or 3 methylation sites are unmethylated in the cell of interest (the cell for which the methylation pattern is being determined), whereas in the second non-identical cell each of the sites are methylated. In some embodiments, in the second non-identical cell is in all other cells. According to another embodiment, each of the 2 or 3 methylation sites are methylated in the cell of interest, whereas in the second non-identical cell each of the sites are unmethylated. In some embodiments, 2 or 3 sites is 2 sites. In some embodiments, 2 or 3 sites is 3 sites. In some embodiments, both sites are methylated in the cell of interest. In some embodiments, all three sites are methylated in the cell of interest. In some embodiments, both sites are unmethylated in the cell of interest. In some embodiments, all three sites are unmethylated in the cell of interest.

The second non-identical cell may be of any source including for example blood cells. In some embodiments, the second non-identical cell is leukocytes. In some embodiments, the second non-identical cell is selected from a blood cell type, vascular endothelial cells and hepatocytes. In some embodiments, a blood cell type is leukocytes. In some embodiments, the tissue or cell type is selected from liver, lung, vascular endothelium, gastro-intestine, B cells, T cells, monocytes, neutrophils, natural killer (NK) cells), and cosinophils. In some embodiments, the cell type is selected from pancreas cells, B cells, breast cells, cardiac cells, colon cells, intestinal cells, blood vessel cells, kidney cells, liver cells, lung cells, bladder cells, endometrial cells, esophageal cells, gallbladder cells, gastric cells, jejunum cells, larynx cells, ovarian cells, pharynx cells, prostate cells, thyroid cells, tongue cells, tonsil cells, erythrocytes, bone marrow cells, fibroblasts, granulocytes, macrophages, brain cells, bone cells, smooth muscle cells, skeletal muscle cells, skin cells, blood cells and immune cells. In some embodiments, the cell type is an endothelial cell. In some embodiments, the cell type is selected from: Acinar, Pancreas; Adipocytes, Abdominal Subcutane.; Alpha, Pancreas; B cells, Blood; Basal epithelial, Breast; Beta, Pancreas; Cardiomyocyte, Heart; Delta, Pancreas; Duct, Pancreas; Endocrine, Colon; Endocrine, Gastric; Endocrine, Jejunum; Endothelium, Aorta; Endothelium, Kidney glomerular; Endothelium, Kidney tubular; Endothelium, Liver; Endothelium, Lung alveolar; Endothelium, Pancreas; Endothelium, Vascular saphenous; Epithelium, Bladder; Epithelium, Colon; Epithelium, Endometrium; Epithelium, Esophagus; Epithelium, Fallopien tubes; Epithelium, Gallbladder; Epithelium, Gastric antrum; Epithelium, Gastric body; Epithelium, Gastric fundus; Epithelium, Jejunum; Epithelium, Kidney glomerular; Epithelium, Kidney tubular; Epithelium, Larynx; Epithelium, Lung alveolar; Epithelium, Lung bronchus; Epithelium, Lung pleural; Epithelium, Ovary; Epithelium, Pharynx; Epithelium, Prostate; Epithelium, Small intestine; Epithelium, Thyroid; Epithelium, Tongue; Epithelium, Tonsil palatine; Epithelium, Tonsil pharyngeal; Erythrocyte progenitors, Bone marrow; Fibroblast, Ad-dermal; Fibroblast, Colon; Fibroblast, Heart; Granulocytes, Blood; Hepatocyte, Liver; Luminal epithelial, Breast; Macrophages, Colon; Macrophages, Liver; Macrophages, Lung alveolar; Macrophages, Lung interstitial; Memory B cells, Blood; Monocytes, Blood; NK, Blood; Naive T cells CD, Blood; Naive T cells CD, Blood; Neuronal, Brain; Oligodendrocytes, Brain; Osteoblasts, Bone; Podocyte, Kidney glomerular; Smooth muscle, Aorta; Smooth muscle, Bladder; Smooth muscle, Bronchial; Smooth muscle, Coronary artery; Smooth muscle, Prostate; Striated muscle, Skeletal muscle; T (CD+) cells, Blood; T central memory CD, Blood; T cytotoxic (CD+) cells, Blood; T effector cell CD, Blood; T effector memory CD, Blood; T effector memory CD, Blood; T helper (CD+) cells, Blood; and keratinocyte, Skin. In some embodiments, T helper cells are CD4 positive. In some embodiments, effector memory cells are CD8 positive. In some embodiments, T effector memory cells are CD4 positive. In some embodiments, T effector cells are CD8 positive. In some embodiments, T cytotoxic cells are CD8 positive. In some embodiments, T central memory cells are CD4 positive. In some embodiments, T cells are CD3 positive. In some embodiments, naïve T cells are CD4 positive. In some embodiments, naïve T cells are CD8 positive. Using this method, the present inventors have identified methylation signatures of 2 or c CpGs in DNA derived from the above enumerated tissues and cell types and showed that these signatures can successfully distinguish between DNA derived from those cells and DNA derived from blood cells and the other cell types.

Thus, according to another aspect of the present invention there is provided a method of determining whether DNA is derived from a cell of interest in a sample, the method comprising: determining the methylation status of two or three methylation sites on a continuous sequence of the DNA, the sequence comprising no more than 167 nucleotides, wherein a methylation status of each of the two or three methylation sites on the continuous sequence characteristic of the cell of interest, is indicative that the DNA is derived from the cell of interest.

It will be appreciated that the method is appropriate for examining if the investigated DNA is derived from a particular cell type or tissue type since the sequences analyzed are specific for particular cell/tissue types. Thus, for example if the investigator wishes to determine if the DNA present in a sample is derived from pancreatic beta cells, he/she needs to analyze sequences which have a methylation pattern characteristic of pancreatic beta cells.

Sequences for identification of specific tissues/cell types are comprised for example in sequences as set forth in SEQ ID NOs: 1-70 and provided in Table 1. SEQ ID NOs: 1-4 comprise sequences which include 2 or 3 methylation sites in a continuous sequence of no more than 167 nucleotides that are unmethylated in liver cells and methylated in other cells (e.g., blood cells). In some embodiments, other cells is all other cells. In some embodiments, other cells are the cells/tissues provided herein. In some embodiments, other cells are blood. SEQ ID NO: 5 comprises a sequence which includes 2 or 3 methylation sites in a continuous sequence of no more than 167 nucleotides that are unmethylated in lung cells and methylated in other cells. SEQ ID NOs: 6-9 comprise sequences which include 2 or 3 methylation sites in a continuous sequence of no more than 167 nucleotides that are unmethylated in universal endothelium and methylated in other cells. SEQ ID NOs: 10-11 comprise sequences which include 2 or 3 methylation sites in a continuous sequence of no more than 167 nucleotides that are unmethylated in GI tract and methylated in other cells. SEQ ID NOs: 12-14 comprise sequences which include 2 or 3 methylation sites in a continuous sequence of no more than 167 nucleotides that are unmethylated in B cells and methylated in other cells. SEQ ID NOS: 15-16 comprise sequences which include 2 or 3 methylation sites in a continuous sequence of no more than 167 nucleotides that are unmethylated in CD8 T cells and methylated in other cells. SEQ ID NOs: 17-18 comprise sequences which include 2 or 3 methylation sites in a continuous sequence of no more than 167 nucleotides that are unmethylated in monocytes and methylated in other cells. SEQ ID NOs: 19-21 comprise sequences which include 2 or 3 methylation sites in a continuous sequence of no more than 167 nucleotides that are unmethylated in neutrophils and methylated in other cells. SEQ ID NOs: 22-23 comprise sequences which include 2 or 3 methylation sites in a continuous sequence of no more than 167 nucleotides that are unmethylated in NK cells and methylated in other cells. SEQ ID NOs: 24-25 comprise sequences which include 2 or 3 methylation sites in a continuous sequence of no more than 167 nucleotides that are unmethylated in T cells and methylated in other cells. SEQ ID NOs: 26-27 comprise sequences which include 2 or 3 methylation sites in a continuous sequence of no more than 167 nucleotides that are unmethylated in Tregs and methylated in other cells. SEQ ID NOs: 28-30 comprise sequences which include 2 or 3 methylation sites in a continuous sequence of no more than 170 nucleotides that are unmethylated in cosinophils and methylated in other cells. SEQ ID NOs: 31-35 comprise sequences which include 2 or 3 methylation sites in a continuous sequence of no more than 167 nucleotides that are unmethylated in pancreas and methylated in other cells. SEQ ID NOs: 36-37 comprise sequences which include 2 or 3 methylation sites in a continuous sequence of no more than 167 nucleotides that are unmethylated in megakaryocytes and methylated in other cells. SEQ ID NOs: 38-49 comprise sequences which include 2 or 3 methylation sites in a continuous sequence of no more than 167 nucleotides that are unmethylated inbrain and methylated in other cells. SEQ ID NOs: 40, 46 and 48 comprise sequences which include 2 or 3 methylation sites in a continuous sequence of no more than 167 nucleotides that are unmethylated in whole brain and methylated in other cells. SEQ ID NOs: 38, 41 and 43 comprise sequences which include 2 or 3 methylation sites in a continuous sequence of no more than 167 nucleotides that are unmethylated in astrocytes and methylated in other cells. SEQ ID NOs: 39, 44 and 45 comprise sequences which include 2 or 3 methylation sites in a continuous sequence of no more than 167 nucleotides that are unmethylated in oligodendrocytes and methylated in other cells. SEQ ID NOs: 42, 47 and 49 comprise sequences which include 2 or 3 methylation sites in a continuous sequence of no more than 167 nucleotides that are unmethylated in neurons and methylated in other cells. SEQ ID NOs: 50-55 comprise sequences which include 2 or 3 methylation sites in a continuous sequence of no more than 167 nucleotides that are unmethylated in heart and methylated in other cells. SEQ ID NOs: 56-60 comprise sequences which include 2 or 3 methylation sites in a continuous sequence of no more than 167 nucleotides that are unmethylated in liver and methylated in other cells. SEQ ID NOs: 61-65 comprise sequences which include 2 or 3 methylation sites in a continuous sequence of no more than 167 nucleotides that are unmethylated in colon and methylated in other cells. SEQ ID NOs: 66-70 comprise sequences which include 2 or 3 methylation sites in a continuous sequence of no more than 167 nucleotides that are unmethylated in endothelial cells and methylated in other cells.

TABLE 1
Sequences of the invention (Chromosome positions are given
relative to human genome build Hg19). In each of these
sequences, there are 2 or 3 specific cytosines whose combined
methylation status is sufficiently informative regarding
the tissue origin of a given DNA molecule. Informative
CpGs are highlighted in the figures (e.g., 1B, 2B).

| | SEQ ID | | |
|---|---|---|---|
| Tissue | NO: | Chromosome | Position Start |
| Hepatocytes | 1 | chr19 | 2790553 |
| Hepatocytes | 2 | chr14 | 70259576 |
| Hepatocytes | 3 | chr12 | 109639335 |
| Hepatocytes | 4 | chr14 | 105964177 |
| Lung | 5 | chr16 | 667119 |
| Vascular endothelial | 6 | chr02 | 16008742 |
| Vascular endothelial | 7 | chr17 | 80803870 |
| Vascular endothelial | 8 | chr19 | 53660138 |
| Vascular endothelial | 9 | chr20 | 3719926 |
| Gastrointestinal | 10 | chr02 | 8849911 |
| Gastrointestinal | 11 | chr19 | 39306152 |
| B cells | 12 | chr11 | 34167796 |
| B cells | 13 | chr17 | 3493576 |
| B cells | 14 | chr11 | 121440655 |
| CD8 | 15 | chr9 | 22436234 |
| CD8 | 16 | chr02 | 87048498 |
| Monocytes | 17 | chr10 | 114911564 |
| Monocytes | 18 | chr11 | 32055198 |
| Neutrophils | 19 | chr11 | 33308278 |
| Neutrophils | 20 | chr09 | 12964828 |
| Neutrophils | 21 | chr08 | 14218008 |
| NK | 22 | chr11 | 64533888 |
| NK | 23 | chr22 | 22035588 |
| T cell | 24 | chr14 | 61800948 |
| T cell | 25 | chr13 | 24825948 |
| Tregs | 26 | chrX | 49117202 |
| Tregs | 27 | chrX | 49118290 |
| Eosinophils | 28 | chr3 | 195974275 |
| Eosinophils | 29 | chr04 | 31230168 |
| Eosinophils | 30 | chr01 | 63412448 |
| Pancreas 1 | 31 | chr16 | 75263456 |
| Pancreas 4 | 32 | chr15 | 102157392 |
| Pancreas 5 | 33 | chr16 | 25228627 |
| Pancreas 6 | 34 | chr13 | 32605843 |
| Pancreas 7 | 35 | chr10 | 133806781 |
| Megakaryocytes 1 | 36 | chr21 | 47808908 |
| Megakaryocytes 2 | 37 | chr21 | 46283807 |
| Astrocyte 1 | 38 | chr16 | 79158122 |
| Oligodendrocyte 1 | 39 | chr06 | 29641034 |
| Whole Brain 1 | 40 | chr16 | 72460052 |
| Astrocyte 2 | 41 | chr01 | 3128518 |
| Neuron 1 | 42 | chr17 | 79253906 |
| Astrocyte 3 | 43 | chr07 | 2656379 |
| Oligodendrocyte 2 | 44 | chr16 | 4521271 |
| Oligodendrocyte 3 | 45 | chr06 | 42046309 |
| Whole Brain 2 | 46 | chr01 | 10239947 |
| Neuron 2 | 47 | chr16 | 310567 |
| Whole Brain 3 | 48 | chr10 | 3283855 |
| Neuron 3 | 49 | chr04 | 4322981 |
| Heart 1 | 50 | chr12 | 124692447 |
| Heart 2 | 51 | chr12 | 109976295 |
| Heart 3 | 52 | chr13 | 117434023 |
| Heart 4 | 53 | chr16 | 68804966 |
| Heart 5 | 54 | chr18 | 76938302 |
| Heart 6 | 55 | chr12 | 124692445 |
| Liver1 | 56 | chr10 | 113943113 |
| Liver2 | 57 | chr17 | 26696304 |
| Liver3 | 58 | chr01 | 145395716 |
| Liver 4 | 59 | chr06 | 160500566 |
| Liver5 | 60 | chr03 | 52864973 |
| Colon1 | 61 | chr02 | 8849910 |
| Colon2 | 62 | chr19 | 2278539 |
| Colon3 | 63 | chr19 | 39306151 |
| Colon4 | 64 | chr01 | 220132243 |
| Colon5 | 65 | chr02 | 8849935 |
| Endothel 1 | 66 | chr02 | 160081740 |
| Endothel 2 | 67 | chr17 | 80803870 |
| Endothel 3 | 68 | chr 20 | 3719897 |
| Endothel 4 | 69 | chr03 | 129214371 |
| Endothel 5 | 70 | ch17 | 82144613 |

By another aspect, there is provided a method of identifying a region of DNA whose methylation signature in a cell type or tissue of interest distinguishes it from a second non-identical cell type or tissue, the method comprising:
a. identifying in DNA of the cell type or tissue of interest a plurality of methylation sites that are each differentially methylated with respect to the second non-identical cell type or tissue of interest;
b. selecting from the plurality of sites at least one site, wherein the at least one site is located in a region comprising at least 4 CpGs within 150 nucleotides upstream and 150 nucleotides downstream of the at least one site; and
c. identifying with the region of DNA a subregion comprising no more than 167 nucleotides and at least 2 methylation sites wherein each of the at least 2 sites are differentially methylated with respect to the second non-identical cell type or tissue of interest;
thereby identifying a region of DNA.

In some embodiments, the DNA is genomic DNA. In some embodiments, the DNA is cell free DNA (cfDNA). In some embodiments, the DNA is circulating DNA. In some embodiments, the DNA is from the cell type or tissue of interest. In some embodiments, the DNA is isolated from the cell type or tissue of interest. In some embodiments, the method further comprises isolating the DNA from the cell type or tissue of interest. In some embodiments, the method further comprises receiving a sample. In some embodiments, the DNA is comprised in a sample. In some embodiments, the sample is a fluid sample. In some embodiments, the fluid is a bodily fluid. In some embodiments, the sample comprises cells of the cell type or tissue of interest.

Samples which may be analyzed are generally fluid samples derived from mammalian subjects and include for example blood, plasma, sperm, milk, urine, saliva or cerebral spinal fluid. In some embodiments, the fluid is selected from the group consisting of blood, plasma, sperm, milk, urine, saliva and cerebral spinal fluid. In some embodiments, the fluid is blood.

Samples which are analyzed typically comprise DNA from at least two cell/tissue sources, as further described herein below.

According to one embodiment, a sample of blood is obtained from a subject according to methods well known in the art. Plasma or serum may be isolated according to methods known in the art.

DNA may be isolated from the blood immediately or within 1 hour, 2 hours, 3 hours, 4 hours, 5 hours or 6 hours. Optionally the blood is stored prior to isolation of the DNA. In some embodiments, a portion of the blood sample is used in accordance with the invention at a first instance of time whereas one or more remaining portions of the blood sample (or fractions thereof) are stored for a period of time for later use.

According to one embodiment, the DNA is cellular DNA (i.e., comprised in a cell).

According to still another embodiment, the DNA is comprised in a shedded cell or non-intact cell.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2nd Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300).

There are also numerous versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

According to another embodiment, the DNA is cell-free DNA. For this method, cell lysis is not performed on the sample. Methods of isolating cell-free DNA from body fluids are also known in the art. For example, Qiaquick kit, manufactured by Qiagen may be used to extract cell-free DNA from plasma or serum.

The sample may be processed before the method is carried out, for example DNA purification may be carried out following the extraction procedure. The DNA in the sample may be cleaved cither physically or chemically (e.g., using a suitable enzyme). Processing of the sample may involve one or more of: filtration, distillation, centrifugation, extraction, concentration, dilution, purification, inactivation of interfering components, addition of reagents, and the like.

It will be appreciated that the present invention contemplates analyzing more than one target sequence (each one comprising at least 2 methylation sites on a continuous sequence of the DNA). Thus, for example 2, 3, 4, 5, 6, 7, 8, 9, 10 or more target sequences (serving as tissue or cell-specific markers) may be analyzed. This may be affected in parallel using the same DNA preparation or on a plurality of DNA preparations.

Methods of determining the methylation status of a methylation site are known in the art include detection of converted DNA molecule and may include the use of bisulfite or enzymatic conversion methods. In one embodiment, the sample can undergo bisulfite conversion. In another embodiment, the conversion of unmethylated cytosines to uracils is accomplished with enzymatic conversion using an enzymatic conversion reaction, e.g., a reaction using a cytidine deaminase (such as APOBEC). After treatment, converted DNA molecules or cfDNA molecules include additional uracils which are not present in the original cfDNA sample. Replication by DNA polymerase of a DNA strand comprising a uracil results in addition of an adenine to the nascent complementary strand instead of the guanine normally added as the complement to a cytosine or methylcytosine. In some embodiments, the converted DNA molecules are converted hypermethylated DNA molecules.

In the bisulfite conversion method, DNA is treated with a reagent such as bisulfite which converts cytosine residues to uracil (which are converted to thymidine following PCR), but leaves 5-methylcytosine residues unaffected. Thus, treatment introduces specific changes in the DNA sequence that depend on the methylation status of individual cytosine residues, yielding single-nucleotide resolution information about the methylation status of a segment of DNA. Various analyses can be performed on the altered sequence to retrieve this information. The objective of this analysis is therefore reduced to differentiating between single nucleotide polymorphisms (cytosines and thymidine) resulting from conversion (i.e. cytosine deamination to uracil).

During the conversion reaction, care should be taken to minimize DNA degradation, such as cycling the incubation temperature.

Bisulfite sequencing relies on the conversion of every single unmethylated cytosine residue to uracil. If conversion is incomplete, the subsequent analysis will incorrectly interpret the unconverted unmethylated cytosines as methylated cytosines, resulting in false positive results for methylation. Only cytosines in single-stranded DNA are susceptible to attack by bisulfite, therefore denaturation of the DNA undergoing analysis is critical. It is important to ensure that reaction parameters such as temperature and salt concentration are suitable to maintain the DNA in a single-stranded conformation and allow for complete conversion.

According to a particular embodiment, an oxidative bisulfite reaction is performed. 5-methylcytosine and 5-hydroxymethylcytosine both read as a C in bisulfite sequencing. Oxidative bisulfite reaction allows for the discrimination between 5-methylcytosine and 5-hydroxymethylcytosine at single base resolution. The method employs a specific chemical oxidation of 5-hydroxymethylcytosine to 5-formylcytosine, which subsequently converts to uracil during bisulfite treatment. The only base that then reads as a C is 5-methylcytosine, giving a map of the true methylation status in the DNA sample. Levels of 5-hydroxymethylcytosine can also be quantified by measuring the difference between bisulfite and oxidative bisulfite sequencing.

Prior to analysis (or concomitant therewith), the bisulfite-treated DNA sequence which comprises the at least four methylation sites may be subjected to an amplification reaction. If amplification of the sequence is required care should be taken to ensure complete desulfonation of pyrimidine residues. This may be affected by monitoring the pH of the solution to ensure that desulfonation is complete.

Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: c2019768118). Non-limiting examples of alternative DNA methylation detection methods are further described below.

Methylation-Specific PCR (MSP), which can be based on a chemical reaction of sodium bisulfite with DNA that converts unmethylated cytosines of CpG dinucleotides to uracil or UpG, followed by traditional PCR. Methylated cytosines will not be converted in this process, and primers are designed to overlap the CpG site of interest, which allows one to determine methylation status as methylated or unmethylated.

The Hpall tiny fragment Enrichment by Ligation-mediated PCR Assay (HELP Assay) compares representations generated by digestion by a restriction enzyme, e.g., Hpall or MspI, of the genome followed by ligation-mediated PCR. Hpall digests 5′-CCGG-3′ sites when the cytosine in the central CG dinucleotide is unmethylated, the Hpall representation is enriched for the hypomethylated fraction of the genome.

Glal hydrolysis and Ligation Adapter Dependent PCR assay (GLAD-PCR assay) can determine R(5mC)GY sites produced in the course of de novo DNA methylation with DNMT3A and DNMT3B DNA methyl transferases. GLAD-PCR. assay do not require bisulfite treatment of the DNA. GLAD-PCR assay uses site-specific methyl-directed DNA-endonucleases (MD DNA endonucleases), which cleave only methylated DNA. and do not cleave unmethylated DNA.

The “Illumina Methylation Assay” measures locus-specific DNA methylation using array hybridization. Bisulfite-treated DNA is hybridized to probes on “BeadChips.” Singlebase base extension with label ed probes is used to determine methylation status of target sites. The Infinium MethylationEPIC BeadChip can interrogate over 850,000 methylation sites across the human genome.

The “Enzymatic Methyl-seq” or “EM-seq” method developed at New England Biolabs provides an alternative to bisulfite modification. This method relies on the ability of APOBEC (e.g., APOBEC-Seq by NEB) to deaminate cytosines to uracils. Then, cytosines are sequenced as thymines and methylated cytosines are sequenced as cytosines.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand-fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the sample DNA together with any additional amplification reaction reagents under conditions which allow amplification of the target sequence.

The terms “forward primer” and “forward amplification primer” are used herein interchangeably and refer to a primer that hybridizes (or anneals) to the target (template strand). The terms “reverse primer” and “reverse amplification primer” are used herein interchangeably and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphospate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favour solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs). RNA target sequences may be amplified by reverse transcribing the mRNA into cDNA, and then performing PCR (RT-PCR), as described above. Alternatively, a single enzyme may be used for both steps as described in U.S. Pat. No. 5,322,770.

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 45.

The denaturation step of a PCR cycle generally comprises heating the reaction mixture to an elevated temperature and maintaining the mixture at the elevated temperature for a period of time sufficient for any double-stranded or hybridized nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

Following denaturation, the reaction mixture is subjected to conditions sufficient for primer annealing to template DNA present in the mixture. The temperature to which the reaction mixture is lowered to achieve these conditions is usually chosen to provide optimal efficiency and specificity, and generally ranges from about 50° C. to about ° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

Following annealing of primer to template DNA or during annealing of primer to template DNA, the reaction mixture is subjected to conditions sufficient to provide for polymerization of nucleotides to the primer's end in a such manner that the primer is extended in a 5′ to 3′ direction using the DNA to which it is hybridized as a template, (i.e., conditions sufficient for enzymatic production of primer extension product). To achieve primer extension conditions, the temperature of the reaction mixture is typically raised to a temperature ranging from about 65° C. to about 75° C., usually from about 67° C. to about 73° C. and maintained at that temperature for a period of time ranging from about 15 seconds to about 20 minutes, usually from about 30 seconds to about 5 minutes.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

According to one embodiment, the primers which are used in the amplification reaction are methylation independent primers. These primers flank the first and last of the at least two methylation sites (but do not hybridize directly to the sites) and in a PCR reaction, are capable of generating an amplicon which comprises all four or more methylation sites. In some embodiments, the primers which are used in the amplification reaction are methylation dependent primers. In some embodiments, the ascertaining comprises amplification. In some embodiments, the amplification is affected using at least one methylation-dependent oligonucleotide (e.g., a primer). This primer will hybridize directly to at least one of the sites and only binds when the site is either methylated or unmethylated. During bisulfite conversion, unmethylated cytosines will be converted to uracil and so the sequence of the methylation dependent oligonucleotide will be different if it is intended to bind methylated or unmethylated DNA. In some embodiments, the site is in the 3′ end of the oligonucleotide. In some embodiments, the 3′ end is the last 10, 9, 8, 7, 6, 5, 4, or 3 nucleotides. Each possibility represents a separate embodiment of the invention. In some embodiments, the 3′ end is the last 5 nucleotides. In some embodiments, the 3′ end is the last 3 nucleotides.

The methylation-independent primers of this aspect of the present invention may comprise adaptor sequences which include barcode sequences. The adaptors may further comprise sequences which are necessary for attaching to a flow cell surface (P5 and P7 sites, for subsequent sequencing), a sequence which encodes for a promoter for an RNA polymerase and/or a restriction site. The barcode sequence may be used to identify a particular molecule, sample or library. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides. The barcode is typically 4-15 nucleotides.

The methylation-independent or dependent oligonucleotide of this aspect of the present invention need not reflect the exact sequence of the target nucleic acid sequence (i.e., need not be fully complementary), but must be sufficiently complementary so as to hybridize to the target site under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target sequence. The conditions are selected such that hybridization of the oligonucleotide to the target site is favored and hybridization to the non-target site is minimized.

Various considerations must be taken into account when selecting the stringency of the hybridization conditions. For example, the more closely the oligonucleotide (e.g., primer) reflects the target nucleic acid sequence, the higher the stringency of the assay conditions can be, although the stringency must not be too high so as to prevent hybridization of the oligonucleotides to the target sequence. Further, the lower the homology of the oligonucleotide to the target sequence, the lower the stringency of the assay conditions should be, although the stringency must not be too low to allow hybridization to non-specific nucleic acid sequences.

As mentioned, the present invention contemplates analyzing more than one target sequence (each one comprising at least four methylation sites on a continuous sequence of the DNA). The sequences may be analyzed individually or as part of a multiplex reaction.

The DNA may be sequenced using any method known in the art-e.g., massively parallel DNA sequencing, sequencing-by-synthesis, sequencing-by-ligation, 454 pyrosequencing, cluster amplification, bridge amplification, and PCR amplification, although preferably, the method comprises a high throughput sequencing method. Typical methods include the sequencing technology and analytical instrumentation offered by Illumina, Ultima Genomics, PacBio, Oxford Nanopore among others.

Other known methods for sequencing include, for example, those described in: Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, l. et al., Dokl Akad Nauk SSSR 303, 1508-1511 (1988); Bains W. & Smith G. C. J. Theor Biol 135, 303-307 (1988); Drnanac, R. et al., Genomics 4, 114-128 (1989); Khrapko, K. R. et al., FEBS Lett 256.118-122 (1989); Pevzner P. A. J Biomol Struct Dyn 7, 63-73 (1989); and Southern, E. M. et al., Genomics 13, 1008-1017 (1992). Pyrophosphate-based sequencing reaction as described, e.g., in U.S. Pat. Nos. 6,274,320, 6,258,568 and 6,210,891, may also be used.

The Illumina sequencing is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Another example of an envisaged sequencing method is pyrosequencing. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed, and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods. The present invention also envisages further developments of these techniques, e.g., further improvements of the accuracy of the sequence determination, or the time needed for the determination of the genomic sequence of an organism etc.

According to one embodiment, the sequencing method comprises deep sequencing. As used herein, the term “deep sequencing” and variations thereof refers to the number of times a nucleotide is read during the sequencing process. Deep sequencing indicates that the coverage, or depth, of the process is many times larger than the length of the sequence under study. In some embodiments, the method further comprises quantitating the amount of cell-free DNA which is derived from the cell type or tissue of interest. In some embodiments, derived from is originates from.

It will be appreciated that any of the analytical methods described herein can be embodied in many forms. For example, it can be embodied on a tangible medium such as a computer for performing the method operations. It can be embodied on a computer readable medium, comprising computer readable instructions for carrying out the method operations. It can also be embodied in electronic device having digital computer capabilities arranged to run the computer program on the tangible medium or execute the instruction on a computer readable medium.

Computer programs implementing the analytical method of the present embodiments can commonly be distributed to users on a distribution medium such as, but not limited to, CD-ROMs or flash memory media. From the distribution medium, the computer programs can be copied to a hard disk or a similar intermediate storage medium. In some embodiments of the present invention, computer programs implementing the method of the present embodiments can be distributed to users by allowing the user to download the programs from a remote location, via a communication network, e.g., the internet. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

Additional methods which rely on the use of bisulfite that may be used to analyze the methylation pattern as described herein are described herein below:

Methylation-sensitive single-nucleotide primer extension: DNA is bisulfite-converted, and bisulfite-specific primers are annealed to the sequence up to the base pair immediately before the CpG of interest. The primer is allowed to extend one base pair into the C (or T) using DNA polymerase terminating dideoxynucleotides, and the ratio of C to T is determined quantitatively. A number of methods can be used to determine this C:T ratio, such as the use of radioactive ddNTPs as the reporter of the primer extension, fluorescence-based methods or Pyrosequencing can also be used. Matrix-assisted laser desorption ionization/time-of-flight (MALDI-TOF) mass spectrometry analysis can be used to differentiate between the two polymorphic primer extension products can be used, in essence, based on the GOOD assay designed for SNP genotyping. Ion pair reverse-phase high-performance liquid chromatography (IP-RP-HPLC) can also be used to distinguish primer extension products.

Base-specific cleavage/MALDI-TOF: This method takes advantage of bisulfite-conversions by adding a base-specific cleavage step to enhance the information gained from the nucleotide changes. By first using in vitro transcription of the region of interest into RNA (by adding an RNA polymerase promoter site to the PCR primer in the initial amplification), RNase A can be used to cleave the RNA transcript at base-specific sites. As RNase A cleaves RNA specifically at cytosine and uracil ribonucleotides, base-specificity is achieved by adding incorporating cleavage-resistant dTTP when cytosine-specific (C-specific) cleavage is desired, and incorporating dCTP when uracil-specific (U-specific) cleavage is desired. The cleaved fragments can then be analyzed by MALDI-TOF. Bisulfite treatment results in cither introduction/removal of cleavage sites by C-to-U conversions or shift in fragment mass by G-to-A conversions in the amplified reverse strand. C-specific cleavage will cut specifically at all methylated CpG sites. By analyzing the sizes of the resulting fragments, it is possible to determine the specific pattern of DNA methylation of CpG sites within the region.

The present inventors further contemplate analyzing the methylation status of the at least two sites including the use of methylation-dependent oligonucleotides.

Methylation dependent oligonucleotides hybridize to either the methylated form of the at least one methylation site or the unmethylated form of the at least one methylation site.

According to one embodiment, the methylation dependent oligonucleotide is a probe. In one embodiment, the probe hybridizes to the methylated site to provide a detectable signal under experimental conditions and does not hybridize to the non-methylated site to provide a detectable signal under identical experimental conditions. In another embodiment, the probe hybridizes to the non-methylated site to provide a detectable signal under experimental conditions and does not hybridize to the methylated site to provide a detectable signal under identical experimental conditions. The probes of this embodiment of this aspect of the present invention may be, for example, affixed to a solid support (e.g., arrays or beads).

According to another embodiment, the methylation dependent oligonucleotide is a primer which when used in an amplification reaction is capable of amplifying the target sequence, when the methylation site is methylated. According to another embodiment, the methylation dependent oligonucleotide is a primer which when used in an amplification reaction is capable of amplifying the target sequence, when the methylation site is unmethylated-see for example International PCT Publication No. WO2013131083, the contents of which are incorporated herein by reference.

The methylation-dependent oligonucleotide of this aspect of the present invention need not reflect the exact sequence of the target nucleic acid sequence (i.e., need not be fully complementary), but must be sufficiently complementary so as to distinguish between a methylated and non-methylated site under the particular experimental conditions. Accordingly, the sequence of the oligonucleotide typically has at least 70% homology, preferably at least 80%, 90%, 95%, 97%, 99% or 100% homology, for example over a region of at least 13 or more contiguous nucleotides with the target sequence. The conditions are selected such that hybridization of the oligonucleotide to the methylated site is favored and hybridization to the non-methylated site is minimized (and vice versa).

By way of example, hybridization of short nucleic acids (below 200 bp in length, e.g. 13-50 bp in length) can be effected by the following hybridization protocols depending on the desired stringency; (i) hybridization solution of 6× SSC and 1% SDS or 3 M TMACI, 0.01 M sodium phosphate (pH 6.8), 1 mM EDTA (pH 7.6), 0.5% SDS, 100 ug/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm (stringent hybridization conditions) (ii) hybridization solution of 6× SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6× SSC, and final wash at 22° C. (stringent to moderate hybridization conditions); and (iii) hybridization solution of 6× SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature at 2.5-3°° C. below the Tm and final wash solution of 6× SSC at 22° C. (moderate hybridization solution).

Oligonucleotides of the invention may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytriyl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

The association between the oligonucleotide and detectable agent can be covalent or non-covalent. Labeled detection probes can be prepared by incorporation of or conjugation to a detectable moiety. Labels can be attached directly to the nucleic acid sequence or indirectly (e.g., through a linker). Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques, and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachments of fluorescent dyes (L. M. Smith et al., Nucl. Acids Res., 1985, 13: 2399-2412) or of enzymes (B. A. Connoly and O. Rider, Nucl. Acids. Res., 1985, 13: 4485-4502); chemical modifications of nucleic acid molecules making them detectable immunochemically or by other affinity reactions (T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl. Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of mono-reactive cisplatin derivatives with the N7 position of guanine moieties in DNA (R. J. Heetebrij et al., Cytogenet. Cell. Genet. 1999, 87: 47-52), psoralen-biotin, which intercalates into nucleic acids and upon UV irradiation becomes covalently bonded to the nucleotide bases (C. Levenson et al., Methods Enzymol. 1990, 184: 577-583; and C. Pfannschmidt et al., Nucleic Acids Res. 1996, 24: 1702-1709), photoreactive azido derivatives (C. Neves et al., Bioconjugate Chem. 2000, 11: 51-55), and DNA alkylating agents (M. G. Sebestyen et al., Nat. Biotechnol. 1998, 16: 568-576).

Any of a wide variety of detectable agents can be used in the practice of the present invention. Suitable detectable agents include, but are not limited to, various ligands, radionuclides (such as, for example, 32P, 35S, 3H, 14C, 125I, 131I, and the like); fluorescent dyes (for specific exemplary fluorescent dyes, see below); chemiluminescent agents (such as, for example, acridinium esters, stabilized dioxetanes, and the like); spectrally resolvable inorganic fluorescent semiconductor nanocrystals (i.e., quantum dots), metal nanoparticles (e.g., gold, silver, copper and platinum) or nanoclusters; enzymes (such as, for example, those used in an ELISA, i.e., horseradish peroxidase, beta-galactosidase, luciferase, alkaline phosphatase); colorimetric labels (such as, for example, dyes, colloidal gold, and the like); magnetic labels (such as, for example, Dynabeads™); and biotin, dioxigenin or other haptens and proteins for which antisera or monoclonal antibodies are available.

In certain embodiments, the inventive detection probes are fluorescently labeled. Numerous known fluorescent labeling moieties of a wide variety of chemical structures and physical characteristics are suitable for use in the practice of this invention. Suitable fluorescent dyes include, but are not limited to, fluorescein and fluorescein dyes (e.g., fluorescein isothiocyanine or FITC, naphthofluorescein, 4′,5′-dichloro-2′,7′-dimethoxy-fluorescein, 6 carboxyfluorescein or FAM), carbocyanine, merocyanine, styryl dyes, oxonol dyes, phycoerythrin, erythrosin, cosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, Spectrum Red™, Spectrum Green™, cyanine dyes (e.g., Cy-3™, Cy-5™, Cy-3.5™, Cy-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like. For more examples of suitable fluorescent dyes and methods for linking or incorporating fluorescent dyes to nucleic acid molecules see, for example, “The Handbook of Fluorescent Probes and Research Products”, 9th Ed., Molecular Probes, Inc., Eugene, Oreg. Fluorescent dyes as well as labeling kits are commercially available from, for example, Amersham Biosciences, Inc. (Piscataway, N.J.), Molecular Probes Inc. (Eugene, Oreg.), and New England Biolabs Inc. (Beverly, Mass.). Another contemplated method of analyzing the methylation status of the sequences is by analysis of the DNA following exposure to methylation-sensitive restriction enzymes-see for example U.S. application Ser. Nos. 20130084571 and 20120003634, the contents of which are incorporated herein.

It will be appreciated that analysis of the methylation status according to methods described herein allows for the accurate determination of cellular source of a DNA molecule, even when the majority of the DNA of the sample is derived from a different cellular source. The present inventors have shown that they are able to determine the cellular source of a particular DNA even when its contribution to the total amount of DNA in the population is less than 1:1000, less than 1:5,000, 1:10,000 or even 1:100,000.

Pathological and disease conditions that involve cell death cause the release of degraded DNA from dying cells into body fluids (blood, plasma, urine, cerebrospinal fluid). Thus, the methods described herein may be used to analyze the amount of cell death of a particular cell population in those body fluids. The amount of cell death of a particular cell population can then be used to diagnose a particular pathological state (e.g., disease) or condition (e.g., trauma).

Thus, according to another aspect of the present invention there is provided a method of detecting death of a cell type or tissue in a subject comprising determining whether cell-free DNA comprised in a fluid sample of the subject is derived from the cell type or tissue, wherein the determining is effected by ascertaining the methylation status of at least two methylation sites on a continuous sequence of the cell-free DNA, the sequence comprising no more than 170 nucleotides, wherein a methylation status of each of the at least two methylation sites on the continuous sequence of the DNA characteristic of the cell type or tissue is indicative of death of the cell type or tissue.

It will be appreciated that death of a particular cell type may be associated with a pathological state—e.g., disease or trauma. The monitoring of the death of a particular cell type may also be used for monitoring the efficiency of a therapeutic regime expected to effect cell death of a specific cell type. The determination of death of a specific cell type may also be used in the clinical or scientific study of various mechanism of healthy or diseased subjects.

Thus, for example measurement of pancreatic beta cell death is important in cases of diabetes, hyperinsulinism and islet cell tumors, and in order to monitor beta cell survival after islet transplantation, determining the efficacy of various treatment regimens used to protect beta cells from death, and determining the efficacy of treatments aimed at causing islet cell death in islet cell tumors. Similarly, the method allows the identification and quantification of DNA derived from dead kidney cells (diagnostic of kidney failure), dead neurons (diagnostic of traumatic brain injury, amyotrophic lateral sclerosis (ALS), stroke, Alzheimer's disease, Parkinson's disease or brain tumors, with or without treatment); dead pancreatic acinar cells (diagnostic of pancreatic cancer or pancreatitis); dead lung cells (diagnostic of lung pathologies including lung cancer); dead adipocytes (diagnostic of altered fat turnover), dead hepatocytes (indicative of liver failure, liver toxicity or liver cancer) dead cardiomyocytes (indicative of cardiac disease, or graft failure in the case of cardiac transplantation), dead skeletal muscle cells (diagnostic of muscle injury and myopathies), dead oligodendrocytes (indicative of relapsing multiple sclerosis, white matter damage in amyotrophic lateral sclerosis, or glioblastoma).

As used herein, the term “diagnosing” refers to determining the presence of a disease, classifying a disease, determining a severity of the disease (grade or stage), monitoring disease progression and response to therapy, forecasting an outcome of the disease and/or prospects of recovery.

The method comprises quantifying the amount of cell-free DNA which is comprised in a fluid sample (e.g., a blood sample) of the subject which is derived from a cell type or tissue. When the amount of cell free DNA derived from the cell type or tissue is above a predetermined level, it is indicative that there is a predetermined level of cell death. When the level of cell death is above a predetermined level, it is indicative that the subject has the disease or pathological state. Determining the predetermined level may be carried out by analyzing the amount of cell-free DNA present in a sample derived from a subject known not to have the disease/pathological state. If the level of the cell-free DNA derived from a cell type or tissue associated with the disease in the test sample is statistically significantly higher than the level of cell-free DNA derived from the same cell type or tissue in the sample obtained from the healthy (non-diseased subject), it is indicative that the subject has the disease. Alternatively, or additionally, determining the predetermined level may be carried out by analyzing the amount of cell-free DNA present in a sample derived from a subject known to have the disease. If the level of the cell-free DNA derived from a cell type or tissue associated with the disease in the test sample is statistically significantly similar to the level of the cell-free DNA derived from a cell type of tissue associated with the disease in the sample obtained from the diseased subject, it is indicative that the subject has the disease.

The severity of disease may be determined by quantifying the amount of DNA molecules having the specific methylation pattern of a cell population associated with the disease. Quantifying the amount of DNA molecules having the specific methylation pattern of a target tissue may be achieved using a calibration curve produced by using known and varying numbers of cells from the target tissue.

According to one embodiment, the method comprises determining the ratio of the amount of cell free DNA derived from a cell of interest in the sample: amount of overall cell free DNA.

According to still another embodiment, the method comprises determining the ratio of the amount of cell free DNA derived from a cell of interest in the sample: amount of cell free DNA derived from a second cell of interest.

The methods described herein may also be used to determine the efficacy of a therapeutic agent or treatment, wherein when the amount of DNA associated with a cell population associated with the disease is decreased following administration of the therapeutic agent, it is indicative that the agent or treatment is therapeutic. In some embodiments, the method further comprises administering a therapeutic agent to a diagnosed subject. In some embodiments, the method further comprises administering or continuing to administer an agent found to be therapeutic.

In some embodiments, the ascertaining is effected by a method comprising contacting the DNA with bisulfite or using an enzymatic method to convert unmethylated cytosines in the DNA to uracil. In some embodiments, the ascertaining is effected by a method comprising amplifying the continuous sequence of DNA. In some embodiments, the amplifying is using methylation-independent oligonucleotides (e.g., primers). In some embodiments, the amplifying is using methylation-dependent oligonucleotides (e.g., primers). In some embodiments, the methylation-independent primers hybridize to the nucleic acid sequence before and after the first and last of the 2 or 3 methylation sites. In some embodiments, before and after is adjacent to.

In some embodiments, identifying a plurality of methylation sites comprises comparing methylation statuses of a plurality of methylation sites with a panel or atlas of methylation statuses. In some embodiments, identifying a subregion comprises comparing the methylation status of the at least 2 methylation sties with a panel or atlas of methylation statuses. In some embodiments, the panel or atlas comprises methylation statuses for the plurality of methylation sites. In some embodiments, the panel or atlas comprises methylation statuses for the at least two methylation sites. In some embodiments, the panel or atlas comprises methylation statuses in DNA. In some embodiments, the DNA is extracted from a plurality of tissues and/or cell types. In some embodiments, the plurality of tissues and/or cell types comprises the second non-identical cell type or tissue. In some embodiments, the panel or atlas comprises methylation statuses from at least 5, 10, 15, 20, 25, 30, 35, 40, 45, 50, 55, 60, 65, or 70 tissues and/or cell types.

In some embodiments, the region comprises no more than 167 nucleotides. In some embodiments, the region comprises at most 167 nucleotides. In some embodiments, nucleotides are basepairs. In some embodiments, the region comprises no more than 167 nucleotides. In some embodiments, the region comprises at most 167 nucleotides. In some embodiments, the region is a continuous sequence. In some embodiments, the region is the subregion. In some embodiments, the method is a method of identifying a subregion. In some embodiments, the region comprises 301 nucleotides. In some embodiments, the region comprises at least 4 CpGs within 301 nucleotides. In some embodiments, the selected at least one site is among the at least 4 CpGs. It will be understood by a skilled artisan that among the many CpGs identified in step (a) only some of them will have at least 3 more CpGs within the surrounding 300 nucleotides. Those that are located in such a region are selected in step (b). In some embodiments, the region stretches from 150 nucleotides upstream of the at least one site to 150 nucleotides downstream of the at least one site. In some embodiments, at least 4 is at least 5. In some embodiments, at least 5 is at least 6.

In some embodiments, step (c) comprises evaluating the at least 4 CpGs and identifying at least 2 that are differentially methylated. In some embodiments, step (c) comprises evaluating the at least 4 CpGs and identifying at least 2 with the same methylation status that are differentially methylated. In some embodiments, the same methylation status is all unmethylation. In some embodiments, the same methylation status is all unmethylated. In some embodiments, the subregion comprises no more than 167 nucleotides. In some embodiments, the subregion comprises at most 167 nucleotides. In some embodiments, the subregion comprises no more than 167 nucleotides. In some embodiments, the subregion comprises at most 167 nucleotides.

In some embodiments, at least 2 is 2. In some embodiments, at least 2 is 3. In some embodiments, at least 2 is 2 or 3. In some embodiments, at least 3 of the at least 4 CpGs are not differentially methylated. In some embodiments, at least 3 is 3. In some embodiments, at least 3 is 4. In some embodiments, 2 or 3 sites are differentially methylated and the other CpGs in the subregion are not differentially methylated. In some embodiments, the at least 2 sites comprise the same methylation status. In some embodiments, the 2 or 3 sites comprises the same methylation status.

By another aspect, there is provided a system for performing a method of the invention, the system comprising aa non-transitory memory device, wherein modules of instruction code are stored, and at least one processor associated with the memory device, and configured to execute the modules of instruction code, whereupon execution of said modules of instruction code, the at least one processor is configured to perform a method of the invention.

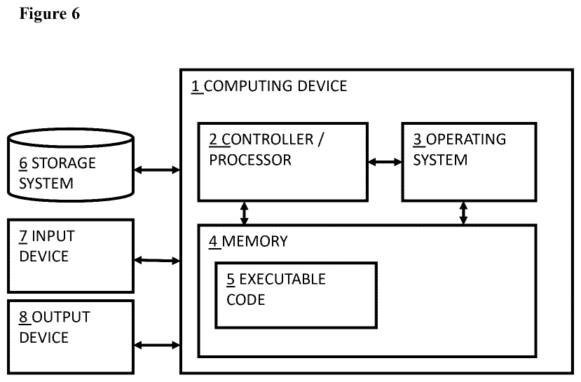

Reference is now made to FIG. 6, which is a block diagram depicting a computing device, which may be included within an embodiment of a system for selecting a therapeutic agent to treat a cancer in a subject, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may selecting a therapeutic agent to treat a cancer in a subject as described herein. Although, for the sake of clarity, a single item of executable code 5 is shown in FIG. 6, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a sample (e.g., a blood sample) taken from one or more patients may be stored in storage system 6, and may be loaded from storage system 6 into memory 4 where it may be processed by processor or controller 2. In some embodiments, some of the components shown in FIG. 6 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

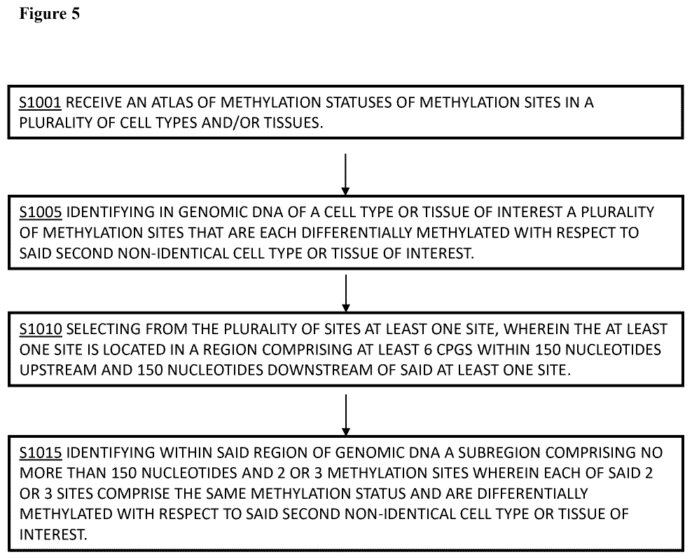

Reference is also made to FIG. 5, which is a flow diagram, depicting an example of a method of identifying a region of DNA whose methylation signature in a cell type or tissue of interest distinguishes it from a second non-identical cell type or tissue, by at least one processor (e.g., processor 2 of FIG. 6) according to some embodiments of the invention.

As shown in optional step S1001, system 10 may receive (e.g., via input device 7 of FIG. 6) one or more (e.g., a plurality of) methylation datasets, each representing the methylation status of CpG sites in DNA of a specific cell type or tissue. System 10 may receive an atlas or panel of methylation datasets from a plurality of cell types and/or tissues. In each methylation dataset there may be the full methylome of the cell type of tissue or only a portion of the methylome corresponding to certain methylation sites. System 10 may also be preloaded with this data.

System 10 may be configured or have a first module for comparing methylation status at a given site across methylation datasets. As shown in step S1005, the first module may be configured to identify in DNA of a specific cell type or tissue (the cell type or tissue of interest) a plurality of methylation sites that are each differentially methylated with respect to a second cell type or tissue. The first module may evaluate a given CpG that is common to at least two databases and see if it is uniquely methylated or unmethylated in a given tissue. The first module can compare not just to a single second tissue or cell type but to all other cell types or tissues inputted into the system (i.e., during step S1001). This can be repeated iteratively until a desired number of sites are identified or alternatively all such sites are identified. That is, not just one CpG can be evaluated but all CpGs for which data is provided for at least 2 cell types/tissues can be evaluated. Alternatively, only CpGs for which data is provided in all datasets are evaluated. Those sites uniquely methylated (whether methylated or unmethylated) are identified and passed to a second module in system 10.

System 10 may include a second module configured to, or as means for, selecting from the identified sites at least one site that has in the 300 nucleotides around it (150 upstream and 150 downstream) at least 5 more CpGs. This combines for a total a region of at most 301 nucleotides that contains at least 6 CpGs including the selected CpG. As shown in step S1010, the second module may be configured to scan up and down 150 nucleotides, for one or more (e.g., each) selected site. The scanning identifies additional CpGs in the region and if there are found to be at least 5 more the region is selected and transferred to a third module.

According to some embodiments, and as elaborated herein, System 10 may include a third module that identifies with the regions subregions of DNA that are not bigger than 150 nucleotides and which contains 2 or more methylation sites that are differentially methylated from a second cell type or tissue. The third module may be the same as or incorporate the first module, as both module compare methylation site status across databases. As shown in step S1015, the third module examines any other CpGs that are within 150 nucleotides of the selected site (this need not be 150 in one direction but can for example be 70 nucleotides upstream and 79 nucleotides downstream for a total of 150 nucleotides) and compares their methylation status to the status in at least one other tissue or cell type. The third module may also compare to a plurality of other tissues/cell types, to all other tissues/cell types for which there is methylation data at the site. Alternatively, only sites for which there is data for all of the tissues/cell types of the atlas are considered. The third module can thus be a combination of the second and first modules in that it scans up and down up to 149 nucleotides using the second module and then compares methylation status using the first module. If a subregion of not than 167 nucleotides is found to have 2 or more uniquely differentially methylated sites, then the subregion is selected. The third module may only select groups of sites that hear the same methylation status; that is sites that are all methylated, or sites that are all unmethylation. Within the 2 or more uniquely differentially methylated sites that would share the same methylation status.

As elaborated herein, embodiments of the invention (e.g., system 10) may provide a practical application for identifying regions that are informative on the origin of DNA. These regions may be used to evaluate cfDNA and/or sequencing data to determine cell death in a subject. This in turn allows for disease diagnosis, and treatment evaluation and recommendation. As such, embodiments of the invention may provide an improvement over currently available systems and methods in the technological field of diagnostics in that they will accurately provide DNA molecule identification with less noise, improved accuracy and most importantly improved sensitivity. Early detection of disease is very difficult, and many detection modalities are invasive. The ability to make accurate diagnostic evaluations from blood or other fluid samples that can be easily obtained greatly improves the diagnostic method, patient classification and therapeutic treatment.

Any of the components described herein may be comprised in a kit. In some embodiments, the kit is for use in a method of the invention. In a non-limiting example, the kit comprises at least one primer pair capable of amplifying a DNA sequence whose methylation status is indicative of a disease, as described hereinabove. According to one embodiment, the primers further comprise barcode sequences and/or sequences which allow for downstream sequencing, as further described herein above. Such primer sequences include for example those that can be used to amplify SEQ ID NOs: 1-30. According to one embodiment, each primer of the primer pair is comprised in a suitable container. According to another embodiment, the kit comprises two primer pairs capable of amplifying two different DNA sequences whose methylation status is indicative of a disease, as described herein above. According to another embodiment, the kit comprises three primer pairs capable of amplifying three different DNA sequences whose methylation status is indicative of a disease, as described herein above. According to another embodiment, the kit comprises four primer pairs capable of amplifying four different DNA sequences whose methylation status is indicative of a disease, as described herein above. According to another embodiment, the kit comprises five or more primer pairs capable of amplifying the five or more different DNA sequences whose methylation status is indicative of a disease, as described herein above.

In another non-limiting example, the kit comprises oligonucleotides which are capable of detecting the methylation status of at least two methylation sites in a nucleic acid sequence, the nucleic acid sequence being no longer than 167 base pairs and comprising at least two methylation sites which are differentially methylated in a first cell of interest with respect to a second cell which is non-identical to the first cell of interest. The kit may comprise one oligonucleotide which is capable of detecting the methylation status of the at least two methylation sites in a nucleic acid sequence. The kit may comprise two oligonucleotides which, in combination are capable of detecting the methylation status of the at least two methylation sites in a nucleic acid sequence. The kit may comprise three oligonucleotides which, in combination are capable of detecting the methylation status of the at least two methylation sites in a nucleic acid sequence. The oligonucleotides of this aspect of the present invention may be labeled with a detectable moiety as further described hereinabove.

Additional components that may be included in any of the above-described kits include at least one of the following components: bisulfite (and other reagents necessary for the bisulfite reaction), a polymerase enzyme, reagents for purification of DNA, MgCl2. In some embodiments, the kit comprises bisulfite. In some embodiments, the kit comprises at least one agent for sequencing of the nucleic acid sequence. In some embodiments, the at least one agent is selected from polymerase, and MgCl2. The kit may also comprise reaction components for sequencing the amplified or non-amplified sequences.

The kits may also comprise DNA sequences which serve as controls. Thus, for example, the kit may comprise a DNA having the same sequence as the amplified sequence derived from a healthy subject (to serve as a negative control) and/or a DNA having the same sequence as the amplified sequence derived from a subject known to have the disease which is being investigated (to serve as a positive control). In addition, the kits may comprise known quantities of DNA such that calibration and quantification of the test DNA may be carried out.

The containers of the kits will generally include at least one vial, test tube, flask, bottle, syringe or other containers, into which a component may be placed, and preferably, suitably aliquoted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented. In some embodiments, instructions are instructions for performing a method of the invention.

It is expected that during the life of a patent maturing from this application many relevant sequencing technologies will be developed (including those that will be able to determine methylation status, without bisulfite treatment: for example, using enzymatic conversion methods such as EM-seq, or using nanopore sequencing) and the scope of the term sequencing is intended to include all such new technologies a priori.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1

International Patent Application WO2015159292 disclosed the use of 4 CpGs in a DNA molecules of approximately 250 nucleotides for use in identifying the cell/tissue type of origin of that DNA. We have now reevaluated the considerations for the design of methylation-based biomarkers, with emphasis on the particular application of plasma cfDNA analysis. We took into account the knowledge regarding the nature of cfDNA (structure of circulating molecules) and the actual tissue origins of human plasma cfDNA. The key facts established in recent years are that cfDNA molecules are typically the size of a nucleosome, i.e., ˜167 nucleotides, and that cfDNA in healthy conditions is derived mostly from hematopoietic cells (˜92%), with the rest derived from vascular endothelial cells and hepatocytes. Other tissues make a negligible contribution to healthy plasma but do contribute under pathologic conditions.

Based on this information, an algorithm was designed that could identify smaller signatures, made up of only 2 or 3 CpGs located within a sequence of less than 167 nucleotides, that could uniquely identify tissues/cell types. The algorithm showed that very often the use of less than 4 CpG, that is 2 or 3 CpGs, in a given marker molecule is possible and even desirable.

Methylation data was acquired for 70 normal human tissues and cell types using the Illumina Infinium Human Methylation 450K array. By comparing the methylation data, the algorithm was able to identify individual CpG sites that were specifically and uniquely methylated or unmethylated in each tissue/cell type. From this list of potential target sites, individual CpGs were selected if they were present in a region of 301 nucleotides (e.g., a region from 150 upstream to 150 downstream of the CpG) that contained greater than 6 CpGs total (regardless of methylation status). All sites that met these criteria were selected. From the selected sites each potential methylation marker region was then assessed for the presence of 2 or 3 CpGs (including the original site) that were within a total size of 167 nucleotides and were all uniquely unmethylated in a given tissue and all fully methylated in all other tissues and cell types. In this way regions of not greater than 167 nucleotides, comprising 2 or 3 CpGs were found that can uniquely identify the 70 tissues/cell types examined. This algorithmic method is summarized in FIG. 5.

FIG. 5 is a flow diagram, depicting an example of a method of identifying a region of genomic DNA comprising no more than 167 nucleotides whose methylation signature uniquely identifies a cell type or tissue of origin, by at least one processor (e.g., processor 2 of FIG. 6) according to some embodiments of the invention. The advantages of using markers identified by this method are exemplified hereinbelow.

These markers provide superior sensitivity compared with longer markers (for example, a 3-CpG marker may capture 90% of hepatocytes, while a 4-CpG marker may capture only 60% of hepatocytes-representing a 50% increase in sensitivity).

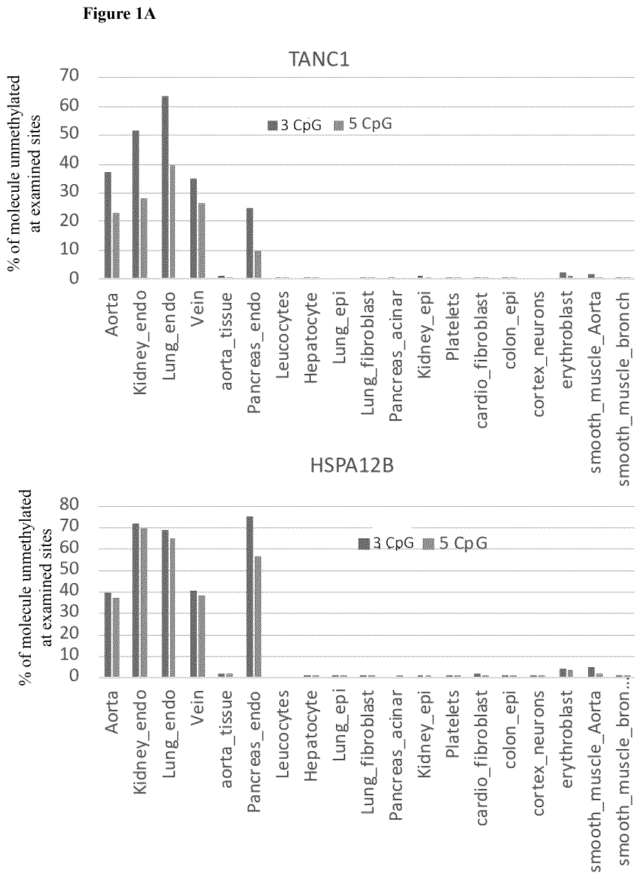

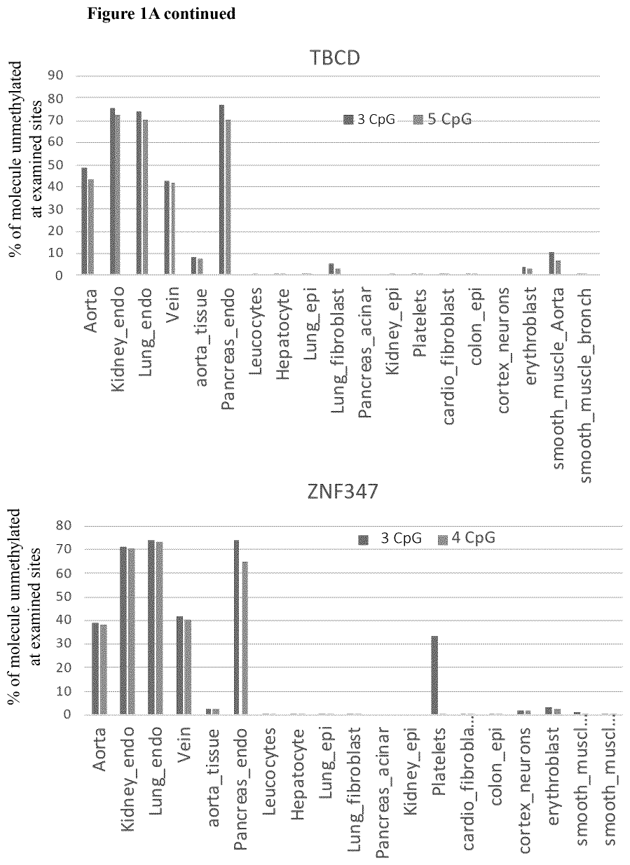

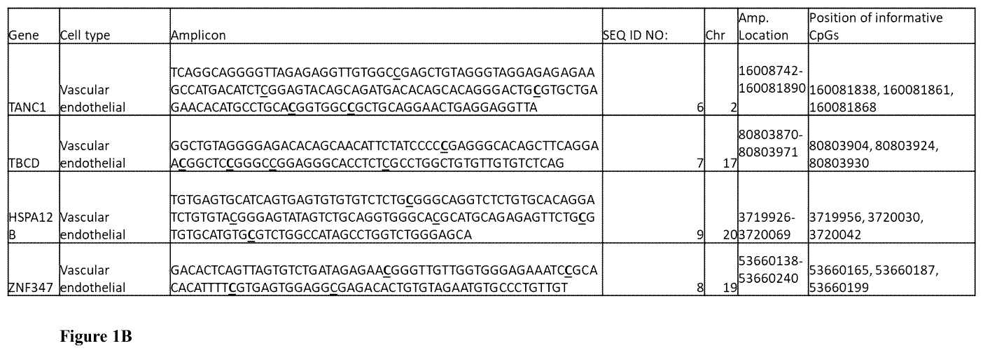

Noise, that is the fraction of molecules (in other tissues) that are unmethylated in the 2 or 3 cytosines contained in the sequence, does not hamper utility of the marker. This is particularly true when analyzing cfDNA from blood, vascular endothelial cells or hepatocytes. cfDNA from these sources is present in a significant proportion (>1%) in plasma. Consequently, identification of elevated levels of cfDNA from these sources requires mostly accuracy and linearity of method and is not sensitive to “noise” in fractions of percent of the molecules from other tissues (as would be the case when searching for ultra-rare molecules, e.g., derived from the heart or brain). FIG. 1A provides an analysis of methylation patterns of 4 different methylation markers of vascular endothelial cell DNA. In all four cases the use of 3 CpGs is just as specific as 4 or 5 CpGs. The 3 informative CpGs are found within the greater cluster of 4 or 5 CpGs. Though some very low noise is observed in some other tissues it is comparable between the use of 3 CpGs and 4 or 5 CpGs, thus indicating that the use of few CpGs does not add noise. The exact sequence of the amplicon and position of the informative CpGs is provided in FIG. 1B.

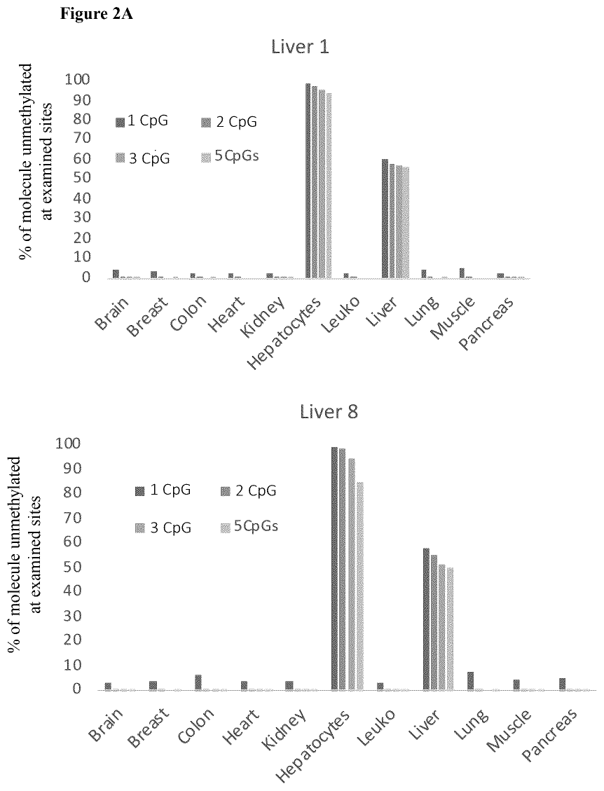

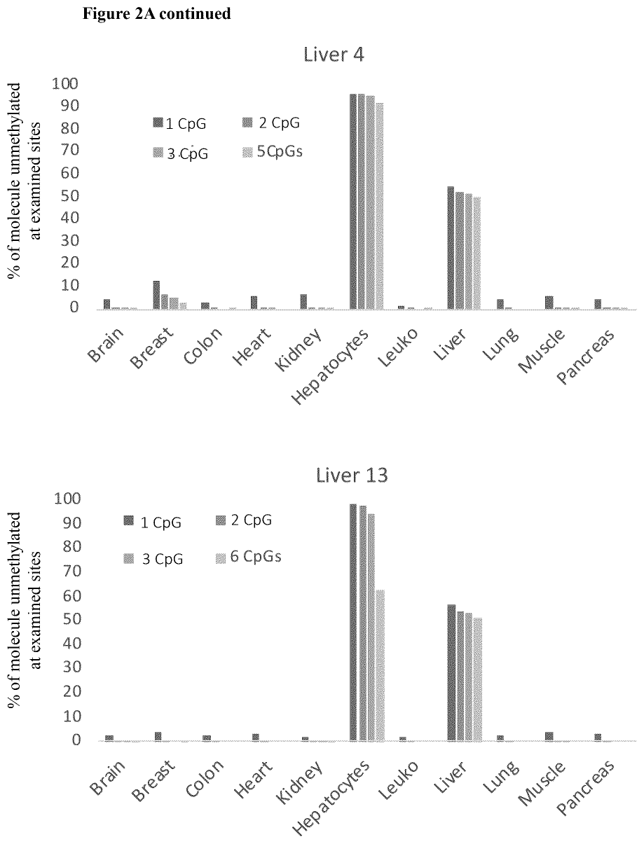

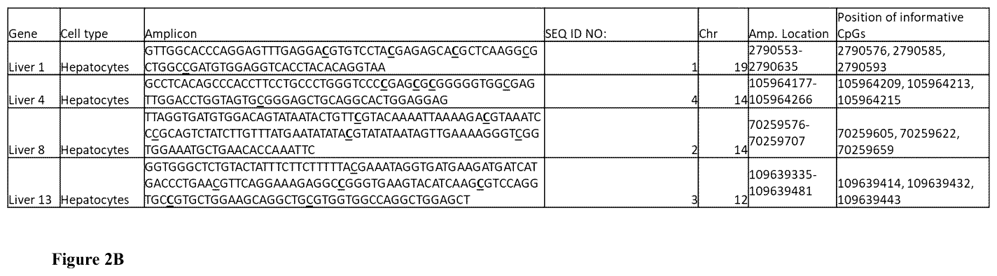

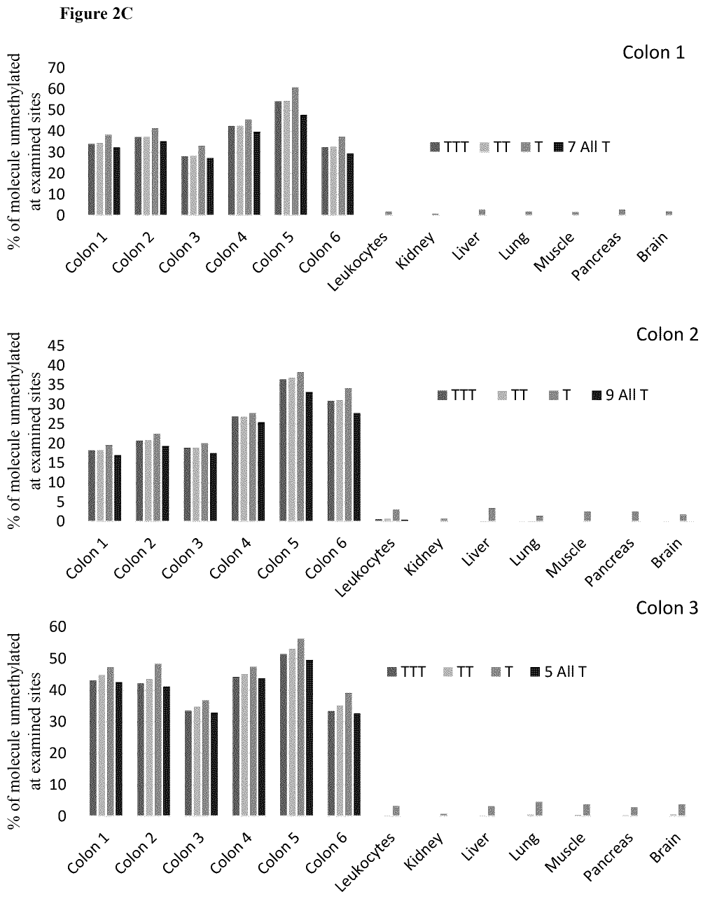

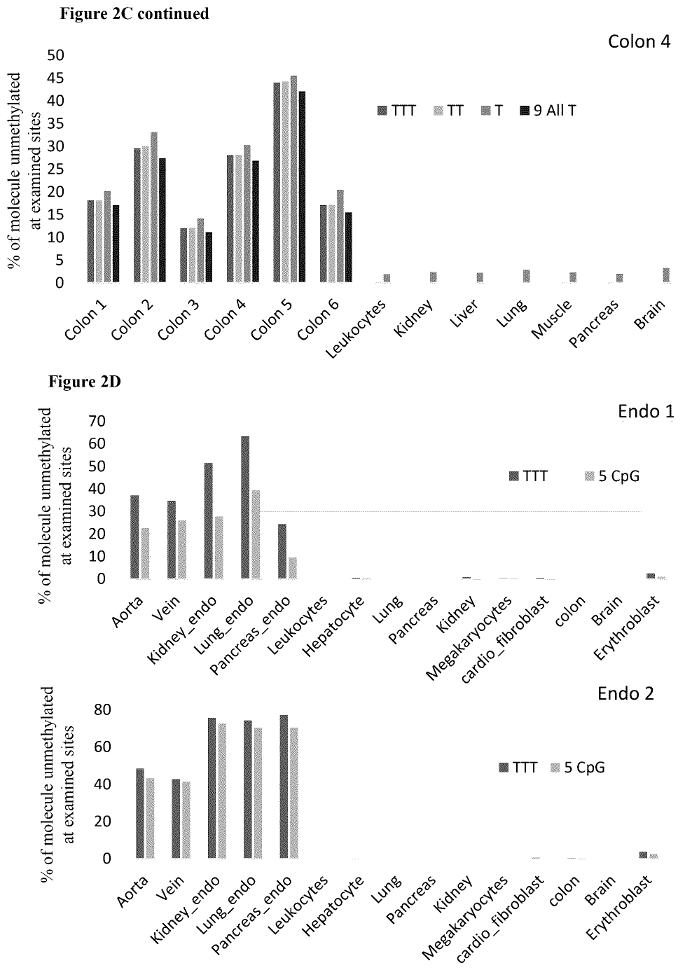

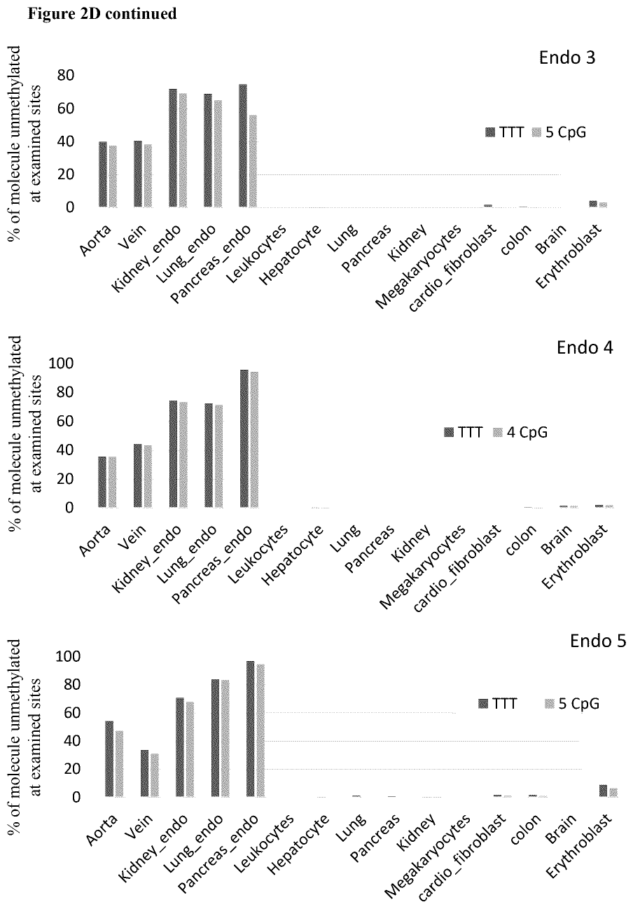

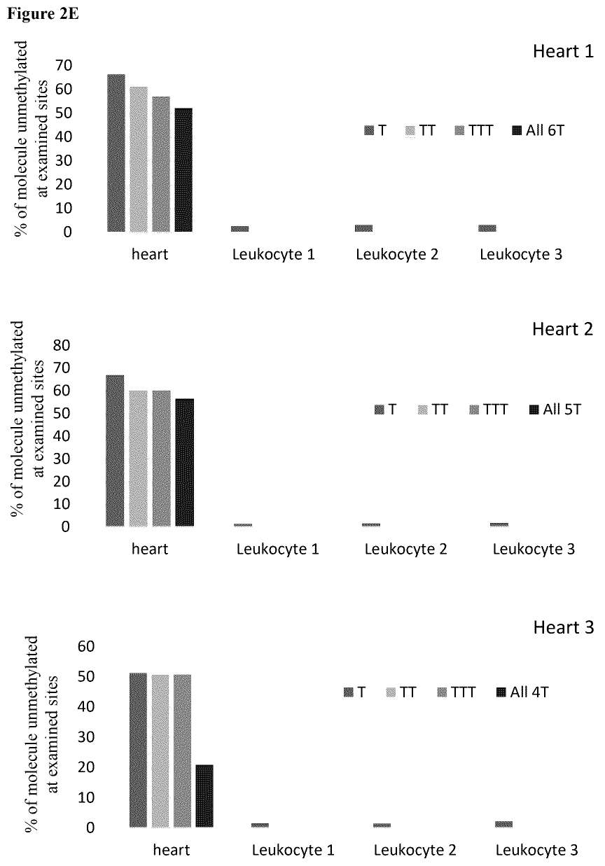

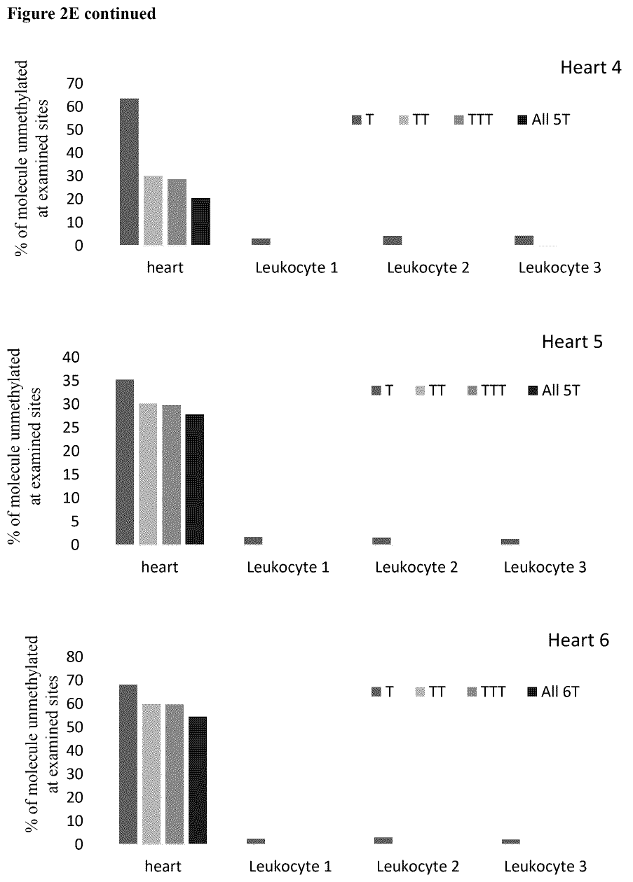

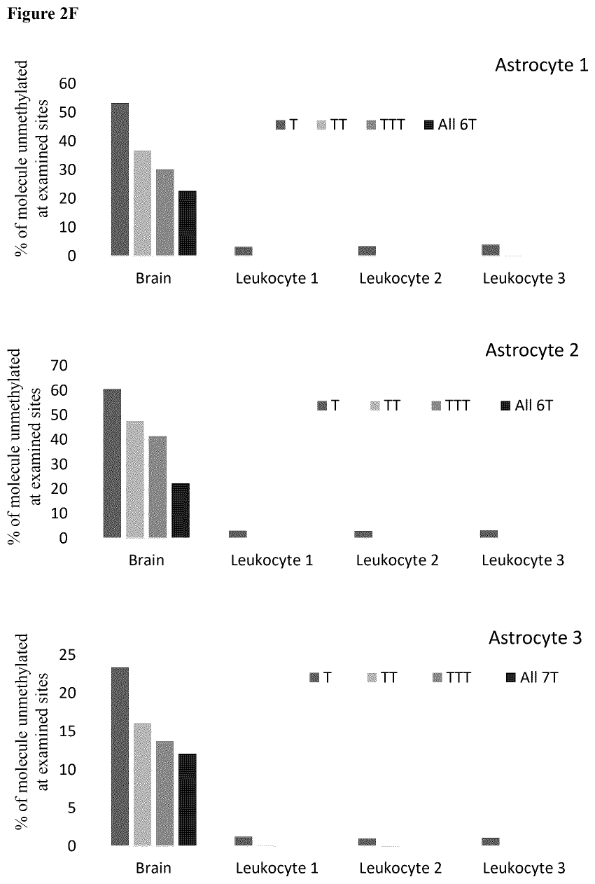

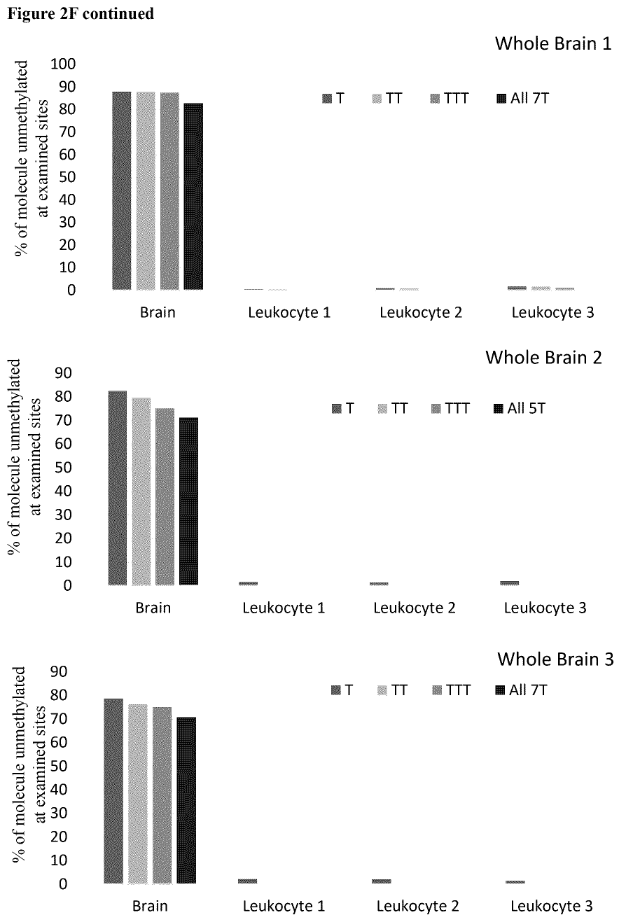

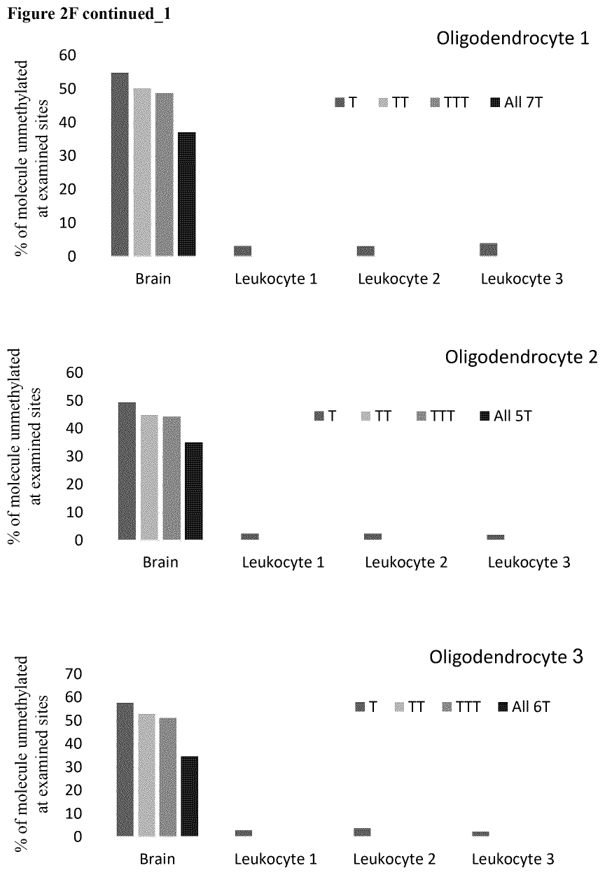

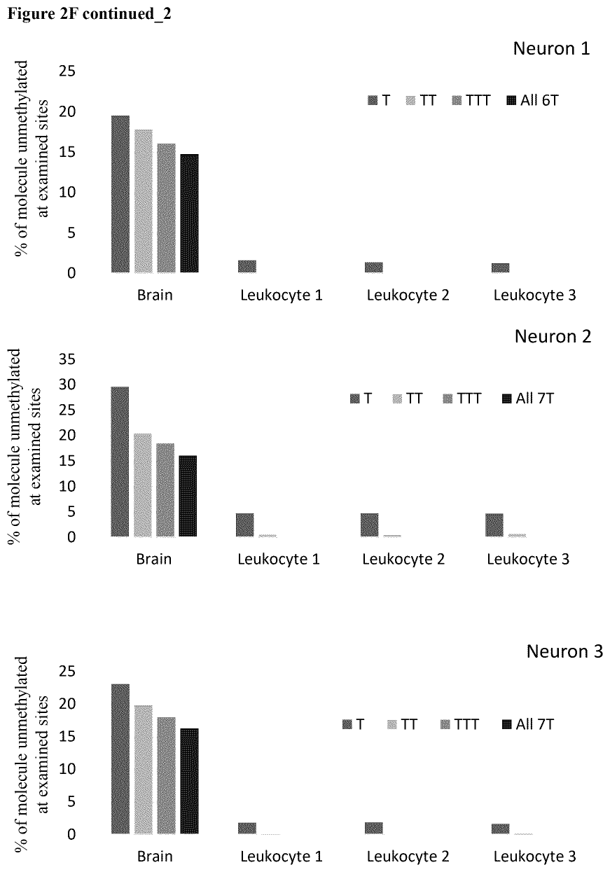

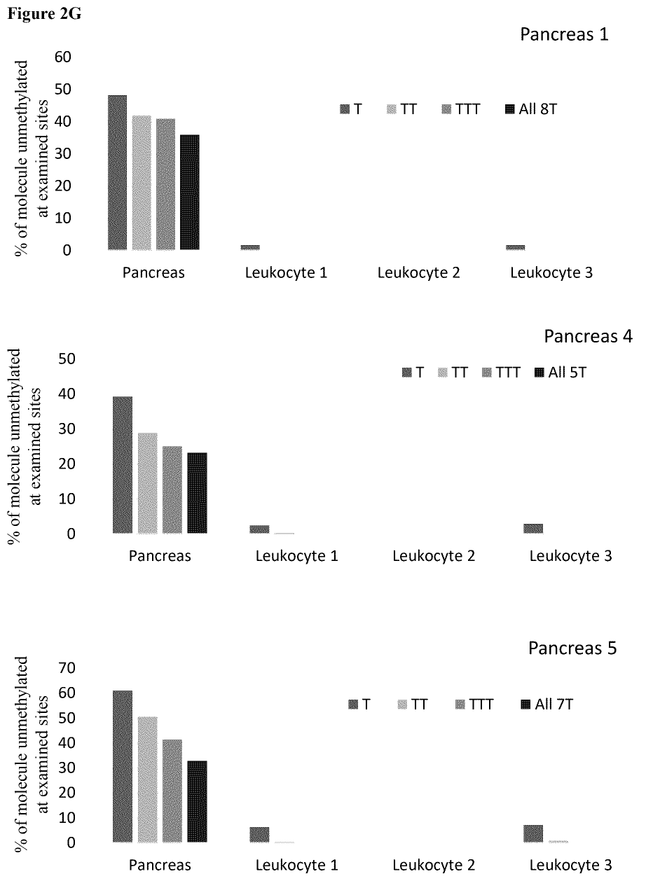

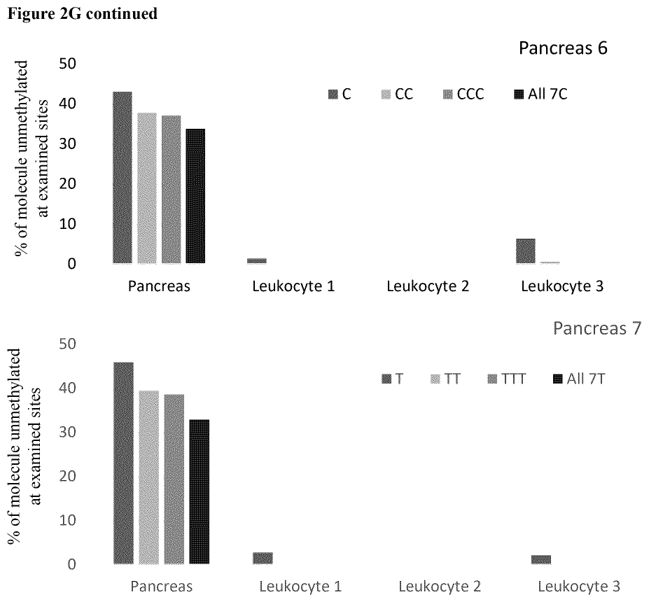

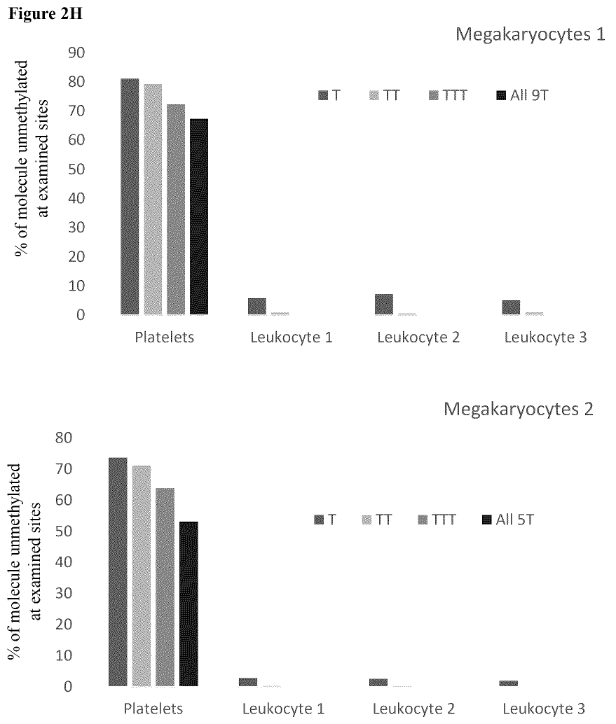

FIG. 2A shows a similar analysis of methylation patterns for 4 hepatocyte DNA markers (SEQ ID NO: 1-4). In this case, 3 or even 2 CpGs are as informative as 5 or 6 CpGs. The exact sequence of the amplicon and position of the informative CpGs is provide in FIG. 2B. Importantly, the methylation status of a single CpG uniformly generates unacceptable noise (>5%) in irrelevant tissues. The 3 informative CpG2 are found with a greater cluster of CpGs (FIG. 2B). FIG. 2C shows a similar analysis of methylation patterns for 4 colon DNA markers (SEQ ID NO: 61-64). The same is shown for 5 endothelial cell markers (FIG. 2D; SEQ ID NO: 66-70), 6 cardiac markers (FIG. 2E; SEQ ID NO: 50-55), 12 neuronal markers (FIG. 2F; SEQ ID NO: 38-49), 5 pancreas markers (FIG. 2G; SEQ ID NO: 31-35) and 2 megakaryocyte makers (FIG. 2H; SEQ ID NO: 36-37). In all cases 2 or 3 CpGs can be as informative or even superior to larger numbers of CpGs. This demonstrates the universality of the method.

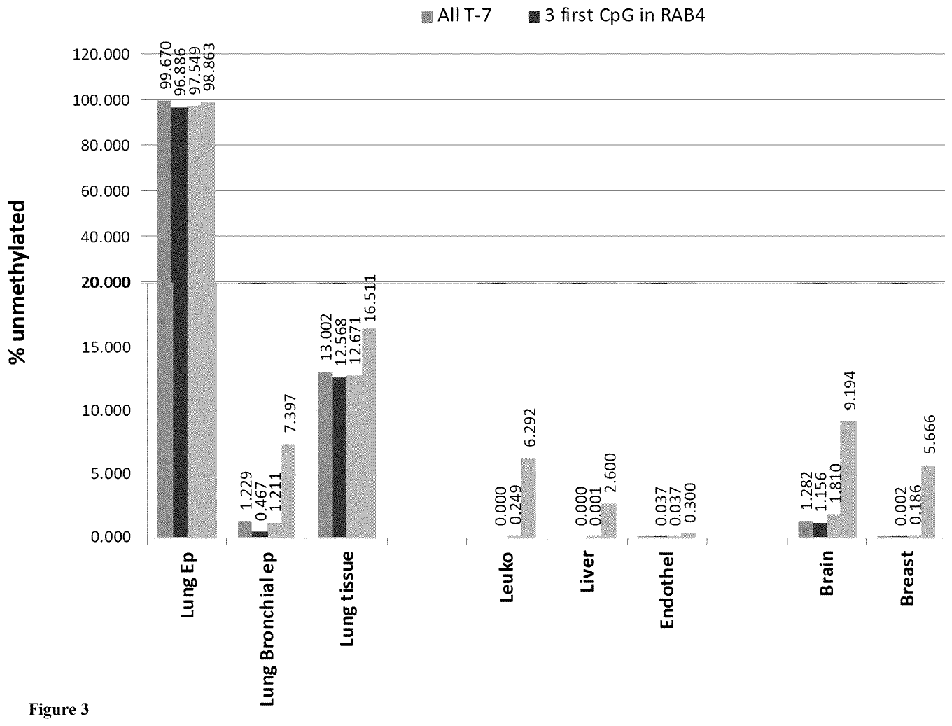

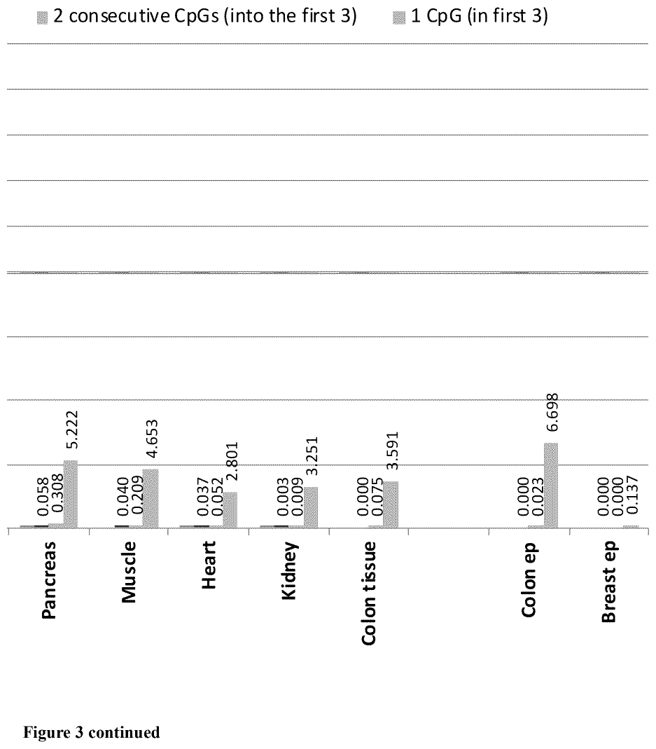

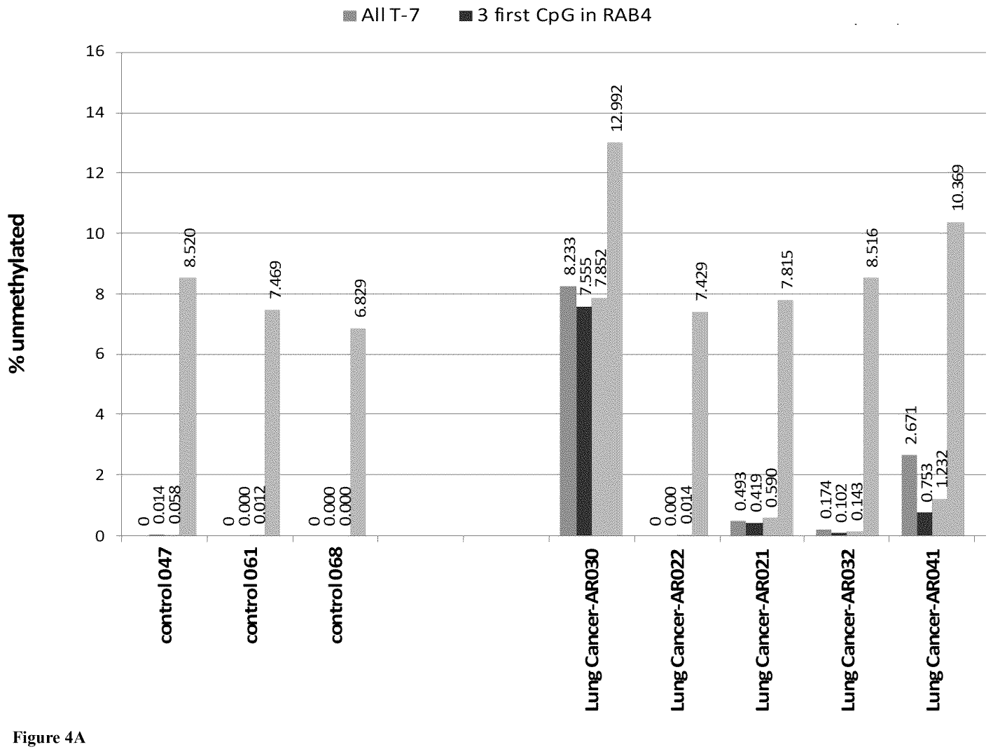

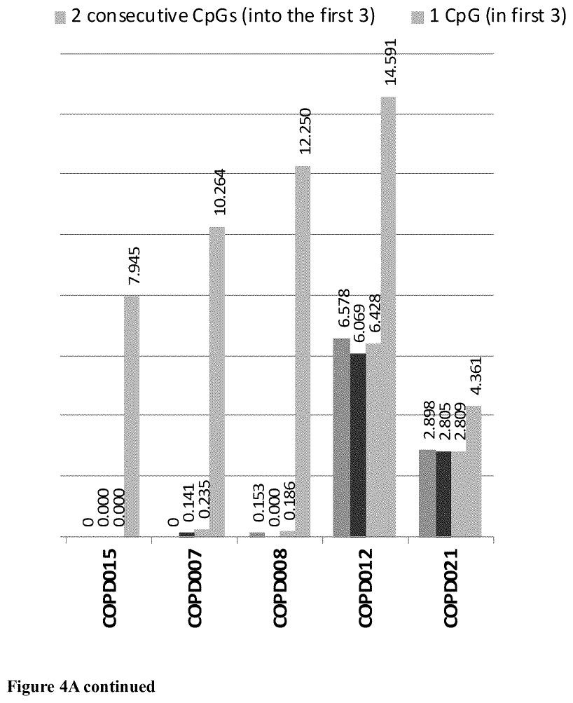

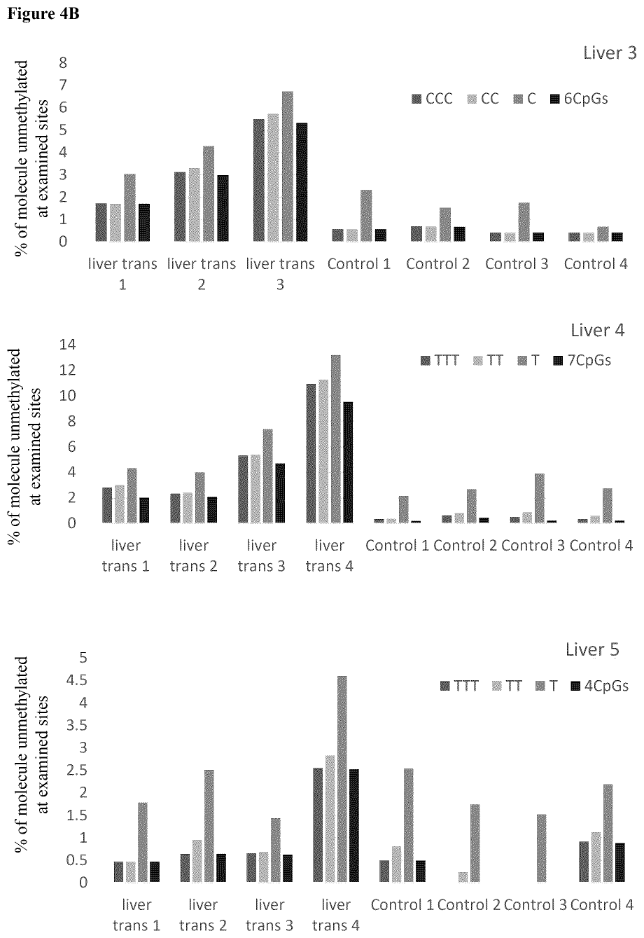

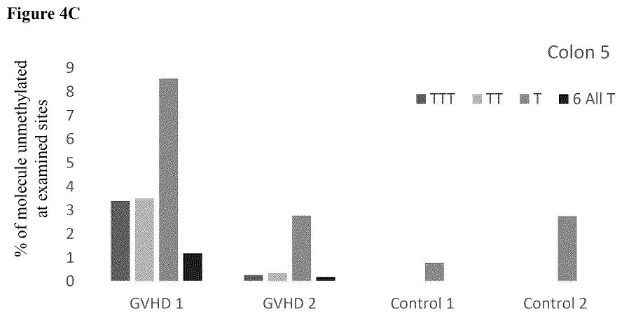

In many cases, even DNA from tissues that typically do not shed detectable cfDNA (and hence the detection of minute amounts of cfDNA requires extreme specificity, without any level of noise from blood), can be identified in cfDNA using markers that consist of 3 or 2 cytosines (but not a single cytosine). FIG. 3 demonstrates this concept for a methylation marker of human lung alveolar cells. From amongst a cluster of 7 CpGs, the use of only 3 or 2 of them was highly specific and informative, however, the use of only a single CpG produced noise from numerous other tissues. Similar detection of other tissues and cell types is presented in FIGS. 2C-2H. Most cell types shown do not shed cfDNA to blood under healthy conditions, but do so in certain pathologies. Of the examples shown, vascular endothelial cells do contribute to about 5-8% of cfDNA normally, and megakaryocyte contribute as much as 30% of cfDNA. FIGS. 4A-4C demonstrate that 2 or 3 cytosines can be used for diagnostic analysis of cfDNA. In FIG. 4A No excessive “noise” is detected in the plasma of healthy individuals when using 2-3 cytosines though the use of only a single cytosine produces unacceptable noise, and there is a clear signal is certain samples from people with lung cancer or COPD (as is expected due to lung cell death). FIG. 4B shows a similar result with a liver marker in plasma samples from healthy control subject and liver transplant recipients during rejection. A single cytosine produces unacceptable noise in the healthy controls making it unsuitable to distinguish them from the transplant recipients. Two or three cytosines are just as good, and in fact often slightly better, than 4 or more cytosines. Detection of graft vs. host disease (GVHD) in a recipient of an allogeneic bone marrow transplant was possible using a colon marker (FIG. 4C). A single cytosine in this marker produced unacceptable signal in the healthy controls, but the use of 2 or 3 cytosines was able to distinguish GVHD from controls. Further, both the 2 and 3 cytosine detection was more than twice as sensitive as the use of all 6 CpGs in the marker region.

Although cfDNA sequencing was used for the analysis shown in FIG. 4, markers based on 2 or 3 cytosines are more convenient targets for detection by quantitative PCR approaches, which are based on short methylation-dependent probes (e.g., digital droplet or real-time PCR). Use of a methylation-dependent probe, as opposed to methylation specific primers, is superior as it is more quantitative and allows for determining the number of source molecules present in the sample.

Our analysis has revealed that markers based on 2-3 CpGs that reside within a <167p locus can be found abundantly in the genome. Such markers detect a higher proportion of molecules from the target tissue than markers that contain 4 or more CpGs (i.e., increased sensitivity for detection of rate DNA molecules from a given cell type as can be seen in FIGS. 1A-1B and 2A-2H).

Some markers of this type, but not all, produce a higher level of background in the DNA of blood, vascular endothelial cells and hepatocytes compared with markers based on 4 or more cytosines (but much lower background than markers based on just one CpG site). Such markers can be identified and avoided through analysis of a panel of genomic DNA samples extracted from multiple tissues.

Many such markers can be found (i.e., a combination of 2 or 3 specific CpGs found in a given DNA stretch, consecutive or separated by other CpGs) which produce a background signal in ˜0.1% of the molecules in irrelevant tissues. This level of background may not be acceptable for applications that seek detection of extremely rate molecules (e.g., present in <0.1% of the total), but is perfectly fine for monitoring contributions from tissues that contribute >1% of the DNA molecules healthy plasma, such as blood, vascular endothelium and liver. In many cases, markers based on 3 or 2 cytosines may be sufficiently specific so as to detect even extremely rare contributions. We demonstrate this principle in data from genomic DNA of multiple tissues and cell types, as well as the plasma of healthy individuals and patients with specific diseases (FIG. 1-4).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of detecting cell type or tissue origin of DNA in a subject comprising determining whether cell-free DNA (cfDNA) comprised in a fluid sample of the subject is derived from the cell type or tissue, wherein said determining is effected by ascertaining the methylation status of two or three methylation sites on a continuous sequence of the cfDNA, said sequence comprising no more than 167 nucleotides, wherein a methylation status of each of said two or three methylation sites on said continuous sequence of the DNA characteristic of said cell type or tissue is indicative of death of the cell type or tissue.

2. The method of claim 1, wherein cfDNA from said cell type or tissue comprises more than 0.1% of the total cfDNA in said fluid sample.

3. The method of claim 2, wherein said cell type or tissue is selected from a blood cell type, vascular endothelial cells and hepatocytes.

4. The method of claim 1, wherein said cell type or tissue is selected from liver, lung, vascular endothelium, gastrointestinal tract, B cells, T cells, monocytes, neutrophils, natural killer (NK) cells and eosinophils.

5. A method of identifying a subregion of genomic DNA comprising no more than 167 nucleotides whose methylation signature in a cell type or tissue of interest distinguishes it from a second non-identical cell type or tissue, the method comprising:
a. identifying in genomic DNA of said cell type or tissue of interest a plurality of methylation sites that are each differentially methylated with respect to said second non-identical cell type or tissue of interest;
b. selecting from said plurality of sites at least one site, wherein said at least one site is located in a region comprising at least 6 CpGs within 167 nucleotides upstream and 167 nucleotides downstream of said at least one site; and
c. identifying with said region of genomic DNA a subregion comprising no more than 167 nucleotides and 2 or 3 methylation sites wherein each of said 2 or 3 sites comprise the same methylation status and are differentially methylated with respect to said second non-identical cell type or tissue of interest;
thereby identifying a subregion of genomic DNA comprising no more than 167 nucleotides.

6. The method of claim 5, wherein:
a. said identifying a plurality of methylation sites comprises comparing methylation statuses of a plurality of methylation sites with a panel or atlas of methylation statuses for said plurality of methylation sites from genomic DNA extracted from a plurality of tissues and/or cell types;
b. said identifying a subregion comprises comparing said methylation status of said 2 or 3 methylation sites with a panel or atlas of methylation statuses for said 2 or 3 methylation sites from genomic DNA extracted from a plurality of tissues and/or cell types; or
c. both.

7. The method of claim 5, wherein said second non-identical cell type or tissue of interest is selected from a blood cell type, vascular endothelial cells and hepatocytes.

8. The method of claim 1, wherein said continuous sequence of the cell-free DNA comprises or consists of a subregion identified by a method of claim 5.

9. The method of claim 1, wherein said continuous sequence of cell-free DNA comprises or consists of a sequence selected from SEQ ID NO: 1-70.

10. The method of claim 1, wherein said fluid is selected from the group consisting of blood, plasma, sperm, milk, urine, saliva and cerebral spinal fluid.

11. The method of claim 10, wherein said sample is a blood sample.

12. The method of claim 1, wherein said ascertaining is effected using at least one methylation-dependent oligonucleotide.

13. The method of claim 1, wherein said ascertaining is effected by:
(a) contacting the DNA in the sample with bisulfite to convert unmethylated cytosines of the DNA to uracils;
(b) amplifying said continuous sequence of DNA using oligonucleotides that hybridize to a nucleic acid sequence adjacent to the first and last of said 2 or 3 methylation sites on said continuous sequence of the DNA; and
(c) sequencing said continuous sequence of DNA.

14. The method of claim 13, wherein said sequencing is deep sequence, next generation sequencing or both.

15. The method of claim 1, further comprising quantitating the amount of cell-free DNA which is derived from said cell type or tissue.

16. The method of claim 1, wherein said method is a computerized method.

17. A system for identifying a subregion of genomic DNA comprising no more than 167 nucleotides whose methylation signature in a cell type or tissue of interest distinguishes it from a second non-identical cell type or tissue, the system comprising a non-transitory memory device, wherein modules of instruction code are stored, and at least one processor associated with the memory device, and configured to execute the modules of instruction code, whereupon execution of said modules of instruction code, the at least one processor is configured to:
identify in genomic DNA of said cell type or tissue of interest a plurality of methylation sites that are each differentially methylated with respect to said second non-identical cell type or tissue of interest;
select from said plurality of sites at least one site, wherein said at least one site is located in a region comprising at least 6 CpGs within 150 nucleotides upstream and 150 nucleotides downstream of said at least one site; and
identify with said region of genomic DNA a subregion comprising no more than 167 nucleotides and 2 or 3 methylation sites wherein each of said 2 or 3 sites comprise the same methylation status and are differentially methylated with respect to said second non-identical cell type or tissue of interest.

18. A kit for identifying the source of DNA in a sample by a method of claim 1, comprising oligonucleotides which are capable of detecting the methylation status of two or three methylation sites in a nucleic acid sequence, said nucleic acid sequence being no longer than 170 base pairs and comprising two or three methylation sites which are differentially methylated in a first cell of interest with respect to a second cell which is non-identical to said first cell of interest.

19. The kit of claim 18, wherein said nucleic acid sequence is comprised in a sequence as set forth in any one of SEQ ID NO: 1-70.

20. The kit of claim 18, further comprising at least one agent for sequencing said nucleic acid sequence, bisulfite or both.

21. (canceled)


SEQUENCE LISTING (70 sequences)

SEQ ID NO: 1 - DNA, 83 bp, Homo sapiens
  source 1..83: mol_type = genomic DNA
gttggcacccaggagtttgaggacgtgtcctacgagagcacgctcaaggcgctggccgatgtggaggtcacctacacaggtaa

SEQ ID NO: 2 - DNA, 132 bp, Homo sapiens
  source 1..132: mol_type = genomic DNA
ttaggtgatgtggacagtataatactgttcgtacaaaattaaaagacgtaaatcccgcagtctatcttgtttatgaatatatacgtatataatagttgaaaagggtcggtggaaatgctgaacaccaaattc

SEQ ID NO: 3 - DNA, 147 bp, Homo sapiens
  source 1..147: mol_type = genomic DNA
ggtgggctctgtactatttcttctttttacgaaataggtgatgaagatgatcatgaccctgaacgttcaggaaagaggccgggtgaagtacatcaagcgtccaggtgccgtgctggaagcaggctgcgtggtggccaggctggagct

SEQ ID NO: 4 - DNA, 90 bp, Homo sapiens
  source 1..90: mol_type = genomic DNA
gcctcacagcccaccttcctgccctgggtccccgagcgcgggggtggcgagttggacctggtagtgcgggagctgcaggcactggaggag

SEQ ID NO: 5 - DNA, 154 bp, Homo sapiens
  source 1..154: mol_type = genomic DNA
ggccctgggaggccagtggccgaggctggtctcgcgggcgctcgctccaggagtggcgcgtcccctcagcgccctgtgcttcctcgcagggatcgactacaagaccaccaccatcctgctggacggccggcgcgtgaagctggagctctggtga

SEQ ID NO: 6 - DNA, 149 bp, Homo sapiens
  source 1..149: mol_type = genomic DNA
tcaggcaggggttagagaggttgtggccgagctgtagggtaggagagagaagccatgacatctcggagtacagcagatgacacagcacagggactgcgtgctgagaacacatgcctgcacggtggccgctgcaggaactgaggaggtta

SEQ ID NO: 7 - DNA, 102 bp, Homo sapiens
  source 1..102: mol_type = genomic DNA
ggctgtaggggagacacagcaacattctatcccccgagggcacagcttcaggaacggctccgggccggagggcacctctcgcctggctgtgttgtgtctcag

SEQ ID NO: 8 - DNA, 103 bp, Homo sapiens
  source 1..103: mol_type = genomic DNA
gacactcagttagtgtctgatagagaacgggttgttggtgggagaaatccgcacacattttcgtgagtggaggcgagacactgtgtagaatgtgccctgttgt

SEQ ID NO: 9 - DNA, 144 bp, Homo sapiens
  source 1..144: mol_type = genomic DNA
tgtgagtgcatcagtgagtgtgtgtctctgcgggcaggtctctgtgcacaggatctgtgtacgggagtatagtctgcaggtgggcacgcatgcagagagttctgcgtgtgcatgtgcgtctggccatagcctggtctgggagca

SEQ ID NO: 10 - DNA, 135 bp, Homo sapiens
  source 1..135: mol_type = genomic DNA
gccctttgtccttgtaggttgacgcctgtgtcagcagagagaggaaagagacgcaggcgatcattctcccgtccttacgtggcagacagggttatttgcgtagattgaccgagatgagtgtcctgcactctgaag

SEQ ID NO: 11 - DNA, 115 bp, Homo sapiens
  source 1..115: mol_type = genomic DNA
gtcagaaggcatagaaacaactgtcatcgcccatcctccctttctgtggatgaggcgggacaaggccggccccctggctggggcctgggacgcgagggctctcagagcttggaga

SEQ ID NO: 12 - DNA, 93 bp, Homo sapiens
  source 1..93: mol_type = genomic DNA
accctctctggctggactgttaaaagcaacgagaggccccggcacacctggaagctggccgcgaattcggcctctgggcctgtgtgtctgtga

SEQ ID NO: 13 - DNA, 115 bp, Homo sapiens
  source 1..115: mol_type = genomic DNA
tcttaaagaagtccccatgggccgcagcctggacgtctgctccgttctccaccaggagggtcaccagggccatgttgcgtctctcgatggcgatgtgcagtgctgtctggcctac

SEQ ID NO: 14 - DNA, 130 bp, Homo sapiens
  source 1..130: mol_type = genomic DNA
aggctgtccctttatttctcagaccggccgacacttagggaaaatagaaaagaacctatgtgaaatatcgggggtgaatttcacccgatatctggctgaatttcccccgatagttactaaagggagggaa

SEQ ID NO: 15 - DNA, 105 bp, Homo sapiens
  source 1..105: mol_type = genomic DNA
tttagtatgattttgagaattttgcgggtagttttggtttgtttttttttacgttttatcgcgatgtgcgtataatagtattgtgatttttgtggtgtattgtag

SEQ ID NO: 16 - DNA, 123 bp, Homo sapiens
  source 1..123: mol_type = genomic DNA
gttaagaaaccaacaggaaaaagaacgcacaactcccagcacagtgctggcgcctgtgaggcactcagccgacgggagctttgttcttcgttgtattgtggcggggaagcaacatggggcctt

SEQ ID NO: 17 - DNA, 109 bp, Homo sapiens
  source 1..109: mol_type = genomic DNA
tgaaggaaatgagagcaaaggtcgtagctgagtgcacgttgaaagaaagcgcggccatcaaccagatccttgggcggagggtaggtgacgcccttctcagggagaaggg

SEQ ID NO: 18 - DNA, 125 bp, Homo sapiens
  source 1..125: mol_type = genomic DNA
ttgccaggttaagcaatctgcaaacgttcgcatgacgtttatgcagccgagtcacatccacattctctccctgtcagttcctttcccgggagctagaaatattgaagtcatttaagtaggagatg

SEQ ID NO: 19 - DNA, 100 bp, Homo sapiens
  source 1..100: mol_type = genomic DNA
tgcaggcacctcagattggggcgtggcgaaacagattgcatttcctagaaggcccccagcgatgtggattgaagcgcaagagtgaggagttggataatca

SEQ ID NO: 20 - DNA, 119 bp, Homo sapiens
  source 1..119: mol_type = genomic DNA
tcctaagaagctcttgtgccaccatcgtgctgaaaagaagagaaagaagcgagtatatttctgacctcttgtgtggcgactgaattgtcggcctcggtctgcacccaggcacccccaga

SEQ ID NO: 21 - DNA, 121 bp, Homo sapiens
  source 1..121: mol_type = genomic DNA
gcctgctttgagatgtgagaaccgtcagtcgggcttggcgggggccgcgcctccatgctcccaggcagcacacattcctcagtctcgactctcgcccgttgtgagcctgcaaggatgccac

SEQ ID NO: 22 - DNA, 80 bp, Homo sapiens
  source 1..80: mol_type = genomic DNA
gacacaagaacagctctgatgtccctccgcccagccgccgccaccaccgaacggcacattcaacctcaaggccacagctg

SEQ ID NO: 23 - DNA, 143 bp, Homo sapiens
  source 1..143: mol_type = genomic DNA
ggaagtaccactgcccagtgctgtttaccgtgttcaccaacaacacccacatcgtggctgtgaggacgaccggcaacgtctacgcctatgaggtgtgtcctcgctccggggcgtggagacagcggtggggagatctctgctgt

SEQ ID NO: 24 - DNA, 123 bp, Homo sapiens
  source 1..123: mol_type = genomic DNA
ggtgttacaggtagggcagagaacgcaagacccagcatctcggctctccagcactgccgccctagcacagggaggtggataacaagcaggcaaacgcacgatgaagaaaatgacaaatgctgg

SEQ ID NO: 25 - DNA, 135 bp, Homo sapiens
  source 1..135: mol_type = genomic DNA
agcacccccattgggttggaccgtgtgggacgccggcggcagatgagagcatccaacggtgagtctcagagtccctttcctttcagagctgctatgggcccatctgaccgtttccagctaacctgaggcagcagg

SEQ ID NO: 26 - DNA, 114 bp, Homo sapiens
  source 1..114: mol_type = genomic DNA
tgggccctgttgtcacagcccccgacttgcccagatttttccgccattgacgtcatggcggccggatgcgccgggcttcatcgacaccacggaggaagagaagagggcagatac

SEQ ID NO: 27 - DNA, 115 bp, Homo sapiens
  source 1..115: mol_type = genomic DNA
ctaggcttggatttcaactctgccgctgcatttcggtgaggccctgagatctctggtcttcaatttgcccttctacactgagcacggagaggcgtggagtagacaagggccaggg

SEQ ID NO: 28 - DNA, 98 bp, Homo sapiens
  source 1..98: mol_type = genomic DNA
ggggcattcctcaccacctcatccacgtagcggcagtgctggactgcgtcatagcgctcattctcgttcatcaccgtgaagcctttgaagttgtgtgt

SEQ ID NO: 29 - DNA, 139 bp, Homo sapiens
  source 1..139: mol_type = genomic DNA
ggagctgttgcagcagctcttcagaacgcctccacccgagcttctgcaaaccctgaccgcagtcgggggcattgggcagctcaccgctgctaaggaggagtctggtggccgaagccgtagtgggagtattgtggaactt

SEQ ID NO: 30 - DNA, 118 bp, Homo sapiens
  source 1..118: mol_type = genomic DNA
tctgagagctgtccacaacagggtcggcgtccaccaacacctcgatctccatggacttattgctcgtgaaggtcatgcgtcccgagatggtgatgacgcagcctgtggagaagggagg

SEQ ID NO: 31 - DNA, 130 bp, Homo sapiens
  source 1..130: mol_type = genomic DNA
gtggcccagctccttgaccctctccaccatgtcctgggccgcggaaggcgatgggtactgcaaggcagcggccttggtggtggccacgatgccgcgcaggaggtcgcacagcaggttgctgtagtgggtc

SEQ ID NO: 32 - DNA, 148 bp, Homo sapiens
  source 1..148: mol_type = genomic DNA
gagaaaatggctctagatcactgcatcttgcagccttgcatctactcgcaatggtcagtcgtaaccatgtgaatgaagcgattcacagaggcacacggggatcaattatagcaaaatggccgaggtgaacatcaccctaatccattaa

SEQ ID NO: 33 - DNA, 146 bp, Homo sapiens
  source 1..146: mol_type = genomic DNA
aactgctgggctctgctctcttcatcttcatcgggtgcctgtcggtcattgagaatgggacggacactgggctgctgcagccggccctggcccacgggctggctttggggctcgtgattgccacgctggggaatatcaggtgagac

SEQ ID NO: 34 - DNA, 111 bp, Homo sapiens
  source 1..111: mol_type = genomic DNA
gctgacatcacaacctgtgacaggcgcatcacgcccggtacctgctcccggccgctgcccgtcctcccagcctctttgtatgccgcagacatggccagccagcaggattgg

SEQ ID NO: 35 - DNA, 115 bp, Homo sapiens
  source 1..115: mol_type = genomic DNA
ttgggatgttttcagttcttgccgctggcaaatcgagctgctgggaacgttcacattcatgtgccagcgtttgtatcgacatgttctttcatttcttgcaggtaaatgccatgga

SEQ ID NO: 36 - DNA, 131 bp, Homo sapiens
  source 1..131: mol_type = genomic DNA
gtcctggtttcctgctagtttccgcacttacagaaggccgagaccggggtttgggctaaatttgtcagctcccgtggacgtggcgctgactcatctcggctggggcgacgttctgagttctgtggcttcct

SEQ ID NO: 37 - DNA, 118 bp, Homo sapiens
  source 1..118: mol_type = genomic DNA
agaagagtttttaagaacatggctcatgccttagaatagaaatcatcgaatacatctttcccggcctcctcaaacctacgctacggtctcgtggaggaagaagagtttttaagaacat

SEQ ID NO: 38 - DNA, 119 bp, Homo sapiens
  source 1..119: mol_type = genomic DNA
gccccctgccccagaactggtcaaacggtgcagagcgctcggcaaatggtcttaaaagcatccgcgcttgcatggaaatgcatttccaatggtgacggggtttgttttattcatggact

SEQ ID NO: 39 - DNA, 143 bp, Homo sapiens
  source 1..143: mol_type = genomic DNA
cctggttttggtgtatcttctggtggcgtttgagctcagactggtcccggaagctcttcccacacacagagcatgaatggggccggtaacccagatggacgcggcggtgacgacttagtccagaagcatcacagtaggtcttg

SEQ ID NO: 40 - DNA, 112 bp, Homo sapiens
  source 1..112: mol_type = genomic DNA
acttgattctgcggcagtggagccagcacagcggatgttttcacacccagcaagacaaaggccgtcgtctccgccatgacactgttccgttccaagcagaggcggggattct

SEQ ID NO: 41 - DNA, 114 bp, Homo sapiens
  source 1..114: mol_type = genomic DNA
gaaagaggggacaataaatagcttcctaccttgcctggataatgggcgagttctccgggtggattaatcctcgcgtcgtctttgggccgtcagtttgggagtgacagtaacaag

SEQ ID NO: 42 - DNA, 141 bp, Homo sapiens
  source 1..141: mol_type = genomic DNA
gagaaggtaatgctgctggaattatggaagagcccgatccacatttacagagccctgcgtgccagagagaaagcaggctcgcgcgcacatgcaggcctccaaatgcatccagcagccgtctgaagtcaatgtttgtgccat

SEQ ID NO: 43 - DNA, 140 bp, Homo sapiens
  source 1..140: mol_type = genomic DNA
tggatagacatggaagtatgtgagcgtggacatccatgcgcacgcgcacaggcacacggggagaaccctcttcctcttccccatccagccctctggtttttggtgctcccagacgtgcgctgagtgcatgagggcctcct

SEQ ID NO: 44 - DNA, 144 bp, Homo sapiens
  source 1..144: mol_type = genomic DNA
atggagcttgagtcacacaggcatgcgcatcggtcagatgtacccttaagattttgcgcgttgcagctgggcccaggggctcatgcctgtaatccagacactttgggaggctgaggcacgcagatctcttgagctcaggagttt

SEQ ID NO: 45 - DNA, 125 bp, Homo sapiens
  source 1..125: mol_type = genomic DNA
agcagatgcatcactgtgaagaagaacgacatgtcggggctgcacctgtcctcccgtcggcatttgacgaaagctccctgaagcggggcagcactctcctcctgagagatttaccatttattgcc

SEQ ID NO: 46 - DNA, 110 bp, Homo sapiens
  source 1..110: mol_type = genomic DNA
ctcagtgccagaactgaaagagcagattcaggcgtggatgagagagaaacagaacagcgatcactaaaccgttccgccgcccaccctctgctagacacagccaaggccaa

SEQ ID NO: 47 - DNA, 104 bp, Homo sapiens
  source 1..104: mol_type = genomic DNA
ctaagcctgaaggtgcaggtctccgagccccagcggagccggcctcgtggaaggacgagggaaagaatgcgtggtgcacggggccatgtgtgtgtgtgggctcc

SEQ ID NO: 48 - DNA, 118 bp, Homo sapiens
  source 1..118: mol_type = genomic DNA
cacatgtgtgcaggctgaataaaatcgtgctgcgggaccacagtgcggggaggcaccgactctgtcattctgtcaacgcaccgcacagtcacgaacatcagcattgacatgaaatgga

SEQ ID NO: 49 - DNA, 119 bp, Homo sapiens
  source 1..119: mol_type = genomic DNA
gcatgactctctgggggctagcgatgaagacgctgcagaatgaaaacgagttagaccagtgatgtaccgcgcttctccacggtagaggcgtgttctcagtttagcaggctggtgttaag

SEQ ID NO: 50 - DNA, 104 bp, Homo sapiens
  source 1..104: mol_type = genomic DNA
gcttggcaatttatccagagaagcaaaatgaccgacaacaacgcacggtgaaacgcagtgttgggaatcgcagatggaagccgagcatttcctctacctgtggg

SEQ ID NO: 51 - DNA, 149 bp, Homo sapiens
  source 1..149: mol_type = genomic DNA
gaatcttgatgtcccctccagagcacgtgaggattaaatgcagttacatatgacgcacccagcgcagcacctggcaccaagtacgcatccgttagtcctcttcctacatttcctcacctttcactttaagaaaacagggctgcccatga

SEQ ID NO: 52 - DNA, 93 bp, Homo sapiens
  source 1..93: mol_type = genomic DNA
agtaatccaggaggctggaatgcgtgtgacaaggacgcttgcttggcgctgctgcctcagcttacatcacgctggaaaatcattgctaaggtc

SEQ ID NO: 53 - DNA, 89 bp, Homo sapiens
  source 1..89: mol_type = genomic DNA
ctgattctcatttcttccatggacacggctaagtgcgggctggtgtgtgagaaagggttcggggcgcgtggttggtggaagttagctcc

SEQ ID NO: 54 - DNA, 121 bp, Homo sapiens
  source 1..121: mol_type = genomic DNA
gtcaccagtttttattgtagtcattctgacgggtgtgcagtgatatcttatcgtggcttcagtttgcgtttctgtgatggccggttgtgttgaacgttgttttgtgtgcttgtttgccatc

SEQ ID NO: 55 - DNA, 109 bp, Homo sapiens
  source 1..109: mol_type = genomic DNA
tggcttggcaatttatccagagaagcaaaatgaccgacaacaacgcacggtgaaacgcagtgttgggaatcgcagatggaagccgagcatttcctctacctgtgggacc

SEQ ID NO: 56 - DNA, 88 bp, Homo sapiens
  source 1..88: mol_type = genomic DNA
ccccttactgcctcaatgttccctagggacccaggcgggctggctggcggcgcctccgcgcgtgcagatacttaggggactgagtcca

SEQ ID NO: 57 - DNA, 108 bp, Homo sapiens
  source 1..108: mol_type = genomic DNA
ggcaccttgaagaggtaggtcttcccctgacagttgatgcgggtgaaggcggcatcgatggggccctcgatgccccagacatctcggatgagcttggggtacccaggc

SEQ ID NO: 58 - DNA, 125 bp, Homo sapiens
  source 1..125: mol_type = genomic DNA
agccttttcataatagctttttgccatgaaggaattcggggtttagagagcgatgggggctgcagagagccaggaagtctcgcggaggactcgggaatcacgagtcctgcctgaaaccttagtgt

SEQ ID NO: 59 - DNA, 94 bp, Homo sapiens
  source 1..94: mol_type = genomic DNA
tgggtgctgccactctgctgacggccacgcatggtttttgtccaggtgactcgcgactcgcgcacctacagcgttggggtgtgcacctttgcag

SEQ ID NO: 60 - DNA, 137 bp, Homo sapiens
  source 1..137: mol_type = genomic DNA
acagtgaagatgtcagtctgctctccctaagtccagcccggggagaaacagcgggggatggtggggaagactagggctacagggctaccctagcggcttcccggaagaagggggcttggccaccaggtaagtgtgtc

SEQ ID NO: 61 - DNA, 135 bp, Homo sapiens; identical to SEQ ID NO: 10
  source 1..135: mol_type = genomic DNA

SEQ ID NO: 62 - DNA, 140 bp, Homo sapiens
  source 1..140: mol_type = genomic DNA
ggcactggggacacctcagagagggtggggctgtggggctcaccttggcgaccaggatgtgccaggcgtcctcgtgcgcgcgcaccacgcggtaatacagggcgtccccgctctctgcgcagggtgagctgtctaggagg

SEQ ID NO: 63 - DNA, 115 bp, Homo sapiens; identical to SEQ ID NO: 11
  source 1..115: mol_type = genomic DNA

SEQ ID NO: 64 - DNA, 147 bp, Homo sapiens
  source 1..147: mol_type = genomic DNA
ggaggccaaggagagggagttgccaacgtaagcgatcactagttccatcacaaagaggaggatgctaacgacgcacatgaggattaaacggaagcttcttccggagtatcgccccatggcgtctcggtgccctggcagttcctctcc

SEQ ID NO: 65 - DNA, 110 bp, Homo sapiens
  source 1..110: mol_type = genomic DNA
ctgtgtcagcagagagaggaaagagacgcaggcgatcattctcccgtccttacgtggcagacagggttatttgcgtagattgaccgagatgagtgtcctgcactctgaag

SEQ ID NO: 66 - DNA, 149 bp, Homo sapiens; identical to SEQ ID NO: 6
  source 1..149: mol_type = genomic DNA

SEQ ID NO: 67 - DNA, 102 bp, Homo sapiens; identical to SEQ ID NO: 7
  source 1..102: mol_type = genomic DNA

SEQ ID NO: 68 - DNA, 144 bp, Homo sapiens; identical to SEQ ID NO: 9
  source 1..144: mol_type = genomic DNA

SEQ ID NO: 69 - DNA, 80 bp, Homo sapiens
  source 1..80: mol_type = genomic DNA
ccaaactgtacaagaggagtgggcacgagaacctcgcgcttgaaatgtacaccgacctctgcatgtttgagtatgccaag

SEQ ID NO: 70 - DNA, 136 bp, Homo sapiens
  source 1..136: mol_type = genomic DNA
tgcagccttcttgggattgtagcctccttcctcgagggaggaagagaaacccaggccgcagcgccctggcccgactggtcctgtccttcggttcattctcccagccacctccccaggcctcttggcaagacccatg